SEQUENCE LISTING (82 sequences)

SEQ ID NO: 1 - protein, 15 aa, Filovirus sp
TIGEWAFWETKKNLT

SEQ ID NO: 2 - protein, 26 aa, Filovirus sp
LILPQAKKDFFSSHPLREPVNATEDP

SEQ ID NO: 3 - protein, 20 aa, Filovirus sp
TTGKLIWKVNPEIDTTIGEW

SEQ ID NO: 4 - protein, 23 aa, Filovirus sp
EIDTTIGEWAFWETKKNLTRKIR

SEQ ID NO: 5 - protein, 28 aa, Filovirus sp
AVSNGPKNISGQSPARTSSDPETNTTNE

SEQ ID NO: 6 - protein, 19 aa, Filovirus sp
NTTNEDHKIMASENSSAMV

SEQ ID NO: 7 - protein, 28 aa, Filovirus sp
ASENSSAMVQVHSQGRKAAVSHLTTLAT

SEQ ID NO: 8 - protein, 39 aa, Filovirus sp
DNSTHNTPVYKLDISEATQVGQHHRRADNDSTASDTPPA

SEQ ID NO: 9 - protein, 25 aa, Filovirus sp
PATTAAGPLKAENTNTSKSADSLDL

SEQ ID NO: 10 - protein, 48 aa, Filovirus sp
ETAGNNNTHHQDTGEESASSGKLGLITNTIAGVAGLITGGRRTRREVI

SEQ ID NO: 11 - protein, 16 aa, Filovirus sp
GLAWIPYFGPAAEGIY

SEQ ID NO: 12 - protein, 23 aa, Filovirus sp
RTFSILNRKAIDFLLQRWGGTCH

SEQ ID NO: 13 - protein, 31 aa, Filovirus sp
DKIDQIIHDFVDKTLPDQGDNDNWWTGWRQW

SEQ ID NO: 14 - protein, 27 aa, Filovirus sp
FLILPQAKKDFFSSHPLREPVNATEDP

SEQ ID NO: 15 - protein, 20 aa, Filovirus sp
TTGKLIWKVNPEIDTTIGEW

SEQ ID NO: 16 - protein, 20 aa, Filovirus sp
EIDTTIGEWAFWETKKTSLE

SEQ ID NO: 17 - protein, 14 aa, Filovirus sp
FKCTVKEGKLQCRI

SEQ ID NO: 18 - protein, 32 aa, Filovirus sp
ASLPKTSGHYDDDDDIPFPGPINDDDNPGHQD

SEQ ID NO: 19 - protein, 21 aa, Filovirus sp
TIPDVVVDPDDGGYGEYQSYS

SEQ ID NO: 20 - protein, 30 aa, Filovirus sp
SENGMSAPDDLVLFDLDEDDEDTKPVPNRS

SEQ ID NO: 21 - protein, 18 aa, Filovirus sp
GQQKNSQKGQHTEGRQTQ

SEQ ID NO: 22 - protein, 18 aa, Filovirus sp
TDNDRRNEPSGSTSPRML

SEQ ID NO: 23 - protein, 18 aa, Filovirus sp
PTVAPPAPVYRDHSEKKE

SEQ ID NO: 24 - protein, 15 aa, Filovirus sp
EQQDQDHIQEARNQD

SEQ ID NO: 25 - protein, 17 aa, Filovirus sp
MPGPELSGWISEQLMTG

SEQ ID NO: 26 - protein, 17 aa, Filovirus sp
DIFCDIENNPGLCYASQ

SEQ ID NO: 27 - protein, 21 aa, Filovirus sp
ATAAATEAYWAEHGQPPPGPS

SEQ ID NO: 28 - protein, 22 aa, Filovirus sp
EHGQPPPGPSLYEESAIRGKIE

SEQ ID NO: 29 - protein, 22 aa, Filovirus sp
ETVPQSVREAFNNLDSTTSLTE

SEQ ID NO: 30 - protein, 26 aa, Filovirus sp
DSTTSLTEENFGKPDISAKDLRNIMY

SEQ ID NO: 31 - protein, 16 aa, Filovirus sp
YMEAIYPARSNSTIAR

SEQ ID NO: 32 - protein, 19 aa, Filovirus sp
LPKYIGLDPVAPGDLTMVI

SEQ ID NO: 33 - protein, 16 aa, Filovirus sp
VITQDCDTCHSPASLP

SEQ ID NO: 34 - protein, 19 aa, Filovirus sp
FPHLFQNPNSTIESPLWAL

SEQ ID NO: 35 - protein, 20 aa, Filovirus sp
ELIYDNNPLKGGLNCNISFD

SEQ ID NO: 36 - protein, 20 aa, Filovirus sp
TGIVSSMHYKLDEVLWEIEN

SEQ ID NO: 37 - protein, 17 aa, Filovirus sp
EDPVEYPADYFRKSKEI

SEQ ID NO: 38 - protein, 14 aa, Filovirus sp
FDVWGNDSNYTKIV

SEQ ID NO: 39 - protein, 22 aa, Filovirus sp
LFQEDGVEEHTKPSYFQAADDS

SEQ ID NO: 40 - protein, 19 aa, Filovirus sp
AEQVEGFIQGPLDDYADEE

SEQ ID NO: 41 - protein, 23 aa, Filovirus sp
GKKSKKLGIAPPPYEEDTSMEYA

SEQ ID NO: 42 - protein, 19 aa, Filovirus sp
PTAAQVQDFGDKWHELPLI

SEQ ID NO: 43 - protein, 16 aa, Filovirus sp
MRVHNNTLINSTSQRV

SEQ ID NO: 44 - protein, 21 aa, Filovirus sp
NTAVKVLAQGDNQVICTQYKT

SEQ ID NO: 45 - protein, 17 aa, Filovirus sp
KIGTGKLGLLINDDETM

SEQ ID NO: 46 - protein, 26 aa, Filovirus sp
LILPQAKKDFFSSHPLREPVNATEDP

SEQ ID NO: 47 - protein, 20 aa, Filovirus sp
TTGKLIWKVNPEIDTTIGEW

SEQ ID NO: 48 - protein, 23 aa, Filovirus sp
EIDTTIGEWAFWETKKNLTRKIR

SEQ ID NO: 49 - protein, 28 aa, Filovirus sp
AVSNGPKNISGQSPARTSSDPETNTTNE

SEQ ID NO: 50 - protein, 19 aa, Filovirus sp
NTTNEDHKIMASENSSAMV

SEQ ID NO: 51 - protein, 28 aa, Filovirus sp
ASENSSAMVQVHSQGRKAAVSHLTTLAT

SEQ ID NO: 52 - protein, 39 aa, Filovirus sp; identical to SEQ ID NO: 8

SEQ ID NO: 53 - protein, 25 aa, Filovirus sp
PATTAAGPLKAENTNTSKSADSLDL

SEQ ID NO: 54 - protein, 48 aa, Filovirus sp; identical to SEQ ID NO: 10

SEQ ID NO: 55 - protein, 31 aa, Filovirus sp; identical to SEQ ID NO: 13

SEQ ID NO: 56 - protein, 27 aa, Filovirus sp
FLILPQAKKDFFSSHPLREPVNATEDP

SEQ ID NO: 57 - protein, 20 aa, Filovirus sp
EIDTTIGEWAFWETKKSTLE

SEQ ID NO: 58 - protein, 20 aa, Filovirus sp
PANEDPVEYPADYFRKSKEI

SEQ ID NO: 59 - protein, 23 aa, Filovirus sp
GKKSKKLGIAPPPYEEDTSMEYA

SEQ ID NO: 60 - protein, 27 aa, Filovirus sp
AVGEIDEIEAQRAEKSNYELFQEDGVE

SEQ ID NO: 61 - protein, 27 aa, Filovirus sp
SMEYAPSAPIDKSYFGVDEMDTYDPNQ

SEQ ID NO: 62 - protein, 16 aa, Filovirus sp
LPQAKKDFFSSHPLRE

SEQ ID NO: 63 - protein, 20 aa, Filovirus sp
  MOD_RES 14: note = K14 is biotinylated
DTTIGEWAFWETKKKNLTRK

SEQ ID NO: 64 - protein, 20 aa, VSV virus
  MOD_RES 5: note = K5 is biotinylated
GKKSKKKLGIAPPPYEEDTS

SEQ ID NO: 65 - protein, 10 aa, influenza virus
YPYDVPDYAG

SEQ ID NO: 66 - protein, 14 aa, polio virus
KEVPALTAVETGAT

SEQ ID NO: 67 - protein, 16 aa, Filovirus sp
LPKYIGLDPVAPGDLT

SEQ ID NO: 68 - protein, 16 aa, Filovirus sp
DDIPFPGPINDDDNPG

SEQ ID NO: 69 - protein, 16 aa, Filovirus sp
EIDTTIGEWAFWETKK

SEQ ID NO: 70 - protein, 16 aa, Filovirus sp
DNSTHNTPVYKLDISE

SEQ ID NO: 71 - protein, 16 aa, Filovirus sp
IAPPPYEEDTSMEYAP

SEQ ID NO: 72 - protein, 12 aa, Filovirus sp
KYIGLDPVAPGD

SEQ ID NO: 73 - protein, 12 aa, Filovirus sp
IPFPGPINDDDN

SEQ ID NO: 74 - protein, 12 aa, Filovirus sp
TIGEWAFWETKK

SEQ ID NO: 75 - protein, 12 aa, Filovirus sp
STHNTPVYKLDI

SEQ ID NO: 76 - protein, 60 aa, Bombali ebolavirus
TFVQLESRFTPQFLVELNETIYIEGKRSNTTGRLIWQVNSRVDTDVGEWAFWENKKTSKR

SEQ ID NO: 77 - protein, 60 aa, Ebola
TYVQLESRFTPQFLLQLNETIYTSGKRSNTTGQLIWKVNPEIDTTIGEWAFWETKKTSLE

SEQ ID NO: 78 - protein, 60 aa, Tai Forest ebolavirus
TYVQLEARFTPQFLVLLNETIYSDNRRSNTTGKLIWKINPTVDTSMGEWAFWENKKTSKK

SEQ ID NO: 79 - protein, 60 aa, Bundibugyo ebolavirus
TYVQLEPRFTPQFLVQLNETIYTNGRRSNTTGTLIWKVNPTVDTGVGEWAFWENKKTSQK

SEQ ID NO: 80 - protein, 60 aa, Zaire ebolavirus
TFVRLDRPHTPQFLFQLNDTIHLHQQLSNTTGRLIWTLDANINADIGEWAFWENKKISPN

SEQ ID NO: 81 - protein, 60 aa, Sudan ebolavirus; identical to SEQ ID NO: 80

SEQ ID NO: 82 - protein, 60 aa, Reston ebolavirus
TYVQLDRPHTPQFLVQLNETLRRNNRLSNSTGRLTWTLDPKIEPDVGEWAFWETKKTFPN

(19) United States
(12) Patent Application Publication
(10) Pub. No.: US 2025/0290927 A1
(43) Pub. Date: Sep. 18, 2025

(54) PEPTIDES AND PEPTIDE MICROARRAYS FOR DETECTION AND DIFFERENTIATION OF ANTIBODY RESPONSES TO EBOLA VIRUS

(71) Applicants: The Trustees of Columbia University in the City of New York, New York, NY (US); The United States of America, as represented by the Secretary, Department of Health and Human servic, Bethesda, MD (US)

(72) Inventors: Nischay MISHRA, New York, NY (US); Walter Ian LIPKIN, New York, NY (US); Sunil Kumar DUBEY, New York, NY (US); Lisa HENSLEY, New York, NY (US)

(21) Appl. No.: 19/220,639

(22) Filed: May 28, 2025

Related U.S. Application Data
(63) Continuation of application No. PCT/US2023/081625, filed on Nov. 29, 2023.
(60) Provisional application No. 63/428,826, filed on Nov. 30, 2022.

(51) Int. Cl. G01N 33/569; G01N 33/543

(52) U.S. Cl. CPC G01N 33/56983; G01N 33/54386; G01N 2333/08; G01N 2469/20

(57) ABSTRACT

Peptides, platforms and methods for detecting antibody responses to filovirus infections, detecting antibody responses to EBOV infection, and detecting antibody responses to vaccination by EBOV vesicular stomatitis virus-based vaccine.

### CROSS-REFERENCE TO RELATED APPLICATIONS

This application is a continuation of PCT International Application No. PCT/US2023/081625, filed Nov. 29, 2023, which claims benefit of U.S. Provisional Application No. 63/428,826, filed Nov. 30, 2022, the contents of each of which are hereby incorporated by reference.

### REFERENCE TO SEQUENCE LISTING

This application incorporates-by-reference nucleotide and/or amino acid sequences which are present in the file named “250528_93597-7371_92225-A-PCT-A_Sequence_Listing_AWG.xml”, which is 71.9 kilobytes in size, and which was created on May 28, 2025, in the IBM-PC machine format, having an operating system compatibility with MS-Windows, which is contained in the xml file filed May 28, 2025 as part of this application.

### BACKGROUND

Improved methods of detecting filovirus infection and for distinguishing between natural infection antibodies and vaccine-induced antibodies are desired.

### SUMMARY OF THE DISCLOSURE

The current disclosure provides compositions, methods, devices, and kits for detecting the exposure to, and infection by, certain viruses and bacteria. Specifically, the current disclosure allows for the rapid differential serological detection of exposure to, and infection by certain viruses and bacteria. In particular, the current disclosure allows for the rapid serological differential detection of exposure to, and infection by a broad range of filoviruses.

Disclosed herein is the identification of peptides that enable specific and sensitive serological detection of adaptive immune responses to a wide range of clinically important high threat pathogens circulating in sub-Saharan Africa on a wide range of platforms including but not limited to phage display, microarray, ELISA, RIA, lateral flow, western blot, and bead-based assays. These assays allow identification of individuals who have been immunized with Ebola virus (EBOV) VSV-EBOV-GP vaccine, and/or infected with Ebola virus (EBOV), Lloviu virus (LLOV), Měnglà virus (MLAV), Bombali virus (BOMV), Bundibugyo virus (BDBV), Reston virus (RESTV), Sudan virus (SUDV), Taï Forest virus (TAFV), and Marburg virus (MARV) and others. Assays based on these discriminatory peptides are not confounded by cross-reactivity between closely related and homologus filoviruses. The importance of this is underscored by two observations: (1) cross reactivity has posed challenges to differential serodiagnosis, efforts to investigate the epidemiology of infection and linkage to disease, and vaccination status with EBOV-VSV vaccine and booster doses; and (2) the only discriminatory platform in current use is a cumbersome plaque reduction neutralization test (PRNT) that is expensive, labor-intensive and requires work with live virus high level biocontainment.

One embodiment of the present disclosure is a peptide, which is reactive with, and specific for an antibody to at least one filovirus pathogen listed in Table 1 or EBOV vaccine. A further embodiment is a collection or set of peptides comprising at least one peptide which is reactive with, and specific for one or more filovirus pathogens in Table 1 or EBOV vaccine.

Yet a further embodiment of the present disclosure are collections or sets of peptides comprising amino acid sequences shifted at least one residue across one or more peptides chosen from the group consisting of the peptides that are reactive with, and specific for an antibody to one or more filovirus pathogens in Table 1 or EBOV vaccine.

Also disclosed herein is the identification of EBOV peptides which can identify and differentiate subjects who have been infected with EBOV, those who have been vaccinated against EBOV, those who are survivors of EBOV, and those who received a vaccination and had a past natural infection. These include:

sixteen EBOV peptides (MGP1-MSGP4) listed in Table 2 and 19 EBOV peptides (MNP1-ML1) listed in Table 3 can be used to identify people who have been infected with EBOV.

sixteen EBOV peptides (MGP1-MSGP4) listed in Table 2, in combination with 9 VSV peptides (VSV1-VSV9) listed in Table 4 and 2 additional VSV peptides (VSV10 and VSV11) listed in Table 6 can be used to identify people who have been vaccinated.

sixteen EBOV peptides (MGP1-MSGP4) listed in Table 2 and 19 EBOV peptides (MNP1-ML1) listed in Table 3 can also identify the EBOV survivors.

combination of 16 EBOV peptides (MGP1-MSGP4) listed in Table 2, 19 EBOV peptides (MNP1-ML1) listed in Table 3, 9 VSV peptides (VSV1-VSV9) listed in Table 4, and 2 additional VSV peptides (VSV10 and VSV11) listed in Table 6 can be used to determine individuals who received vaccination and had a past natural infection both.

A further embodiment of the present disclosure are collections or sets of peptides comprising amino acid sequences shifted at least one residue across one or more peptides chosen from the group consisting of the peptides chosen from the group consisting of the peptides listed in Tables 2, 3, 4 and 6 and combinations thereof.

In non-limiting examples, the antibodies can be detected using any number of immunodetection techniques, which include but are not necessarily limited to microarrays, ELISA, RIA, lateral flow, western blot, bead-based assays, dipstick type of assay or a SNAP test, multiplex antibody detection techniques of various kinds, or any modification of such assays that are suitable for detecting antibodies of interest using the disclosed peptides.

In certain embodiments, peptides are attached to or immobilized on a solid support. In one embodiment, the peptides are attached to a solid support through a metallic nanolayer. In certain embodiments, the solid support is a bead (e.g., a colloidal particle, metallic nanoparticle or nanoshell, or latex bead), a flow path in a lateral flow immunoassay device (e.g., a porous membrane), a blot (e.g., Western blot, a slot blot, or dot blot), a flow path in an analytical or centrifugal rotor, or a tube or well (e.g., in a plate suitable for an ELISA assay or microarray). In certain embodiments, peptides are isolated (e.g., synthetic and/or purified) peptides. In certain embodiments, peptides are conjugated to a ligand. For example, in certain embodiments, the peptides are biotinylated. In other embodiments, the peptides are conjugated to streptavidin, avidin, or neutravidin. In other embodiments, the peptides are conjugated to a carrier protein (e.g., serum albumin, keyhole limpet hemocyanin (KLH), or an immunoglobulin Fc domain).

In one embodiment, the immunodetection technique is in the form of a programmable peptide array.

Also described herein is a sensitive, highly multiplexed, microarray-based assay that enables the discrimination of antibody responses to linear epitopes specific to several high threat filovirus pathogens circulating in sub-Saharan Africa including those listed in Table 1 as well as linear epitopes specific to EBOV vaccine.

A further embodiment is a peptide microarray comprising peptides which are reactive with, and specific for EBOV infection and/or EBOV vaccine chosen from the group consisting of the peptides listed in Tables 2, 3, 4 and 6 and combinations thereof, and/or a collection or set of peptides shifted at least one residue across at least one peptide chosen from the group consisting of the peptides listed in Tables 2, 3, 4 and 6 and combinations thereof.

In another aspect, the disclosure provides devices. In certain embodiments, the devices are useful for performing an immunoassay. For example, in certain embodiments, the device is a lateral flow immunoassay device. In other embodiments, the device is an analytical or centrifugal rotor. In other embodiments, the device is a tube or a well, e.g., in a plate suitable for an ELISA assay or a microarray. In still other embodiments, the device is an electrochemical, optical, or opto-electronic sensor.

In certain embodiments, the device comprises at least one disclosed peptide. In other embodiments, the device comprises a collection or set of the disclosed peptides as described herein. In certain embodiments, the peptides are attached to or immobilized upon the device.

The disclosure also provides methods of using the disclosed peptides and peptide microarrays.

In one embodiment, the disclosure provides methods of detecting in a sample an antibody to an epitope of one or more filovirus pathogens in Table 1 and/or EBOV vaccine. In certain embodiments, the methods comprise contacting a sample with one or more disclosed peptides which are reactive with, and specific for antibodies of one or more filovirus pathogens in Table 1 or EBOV vaccine and detecting formation of an antibody-peptide complex comprising said peptide(s), wherein formation of said complex is indicative of the presence of an antibody to an epitope in said sample.

In one embodiment, the disclosure provides a method for the serological detection of exposure to and/or infection by one or more filovirus pathogens in Table 1 and/or immunization by EBOV vaccine, comprising the use of one or more disclosed peptides which are reactive with, and specific for antibodies of one or more filovirus pathogens in Table 1 or EBOV vaccine.

Yet a further embodiment is a method for the differential serological detection of exposure to and/or infection by one or more filovirus pathogens in Table 1 and/or immunization by EBOV vaccine, comprising the use of a one or more disclosed peptides which are reactive with, and specific for antibodies of one or more pathogens in Table 1 or EBOV vaccine.

In a further embodiment, the disclosure provides for a method of detecting in a sample an antibody to an epitope of one or more filovirus pathogens in Table 1 and/or EBOV vaccine using a peptide microarray. In certain embodiments, the method comprises contacting a sample with a peptide microarray comprising one or more disclosed peptides which are reactive with, and specific for antibodies of one or more filovirus pathogens in Table 1 or EBOV vaccine and detecting formation of an antibody-peptide complex comprising said peptide(s), wherein formation of said complex is indicative of the presence of an antibody to an epitope in said sample.

A further embodiment is a method for the serological detection of exposure to and/or infection by one or more filovirus pathogens in Table 1, comprising the use of a peptide microarray comprising one or more disclosed peptides which are reactive with, and specific for one or more pathogens in Table 1 or EBOV vaccine.

A further embodiment is a method for the differential serological detection of exposure to and/or infection by one or more filovirus pathogens in Table 1 and/or immunization by EBOV vaccine, comprising the use of a peptide microarray comprising one or more disclosed peptides which are reactive with, and specific for antibodies of one or more filovirus pathogens in Table 1 or EBOV vaccine.

A further embodiment is a method of identifying a subject who has been infected with EBOV and/or survived EBOV comprising the use of at least one peptide chosen from the group consisting of the peptides listed in Tables 2 and 3 and combinations thereof and/or a collection or set of peptides shifted at least one residue across at least one peptide chosen from the group consisting of the peptides listed in Tables 2 and 3 and combinations thereof.

A further embodiment is a method of identifying a subject who has been vaccinated against EBOV, comprising the use of at least one peptide chosen from the group consisting of the peptides listed in Tables 2, 4 and 6 and combinations thereof and/or a collection or set of peptides shifted at least one residue across at least one peptide chosen from the group consisting of the peptides listed in Tables 2, 4 and 6 and combinations thereof.

A further embodiment is a method of identifying subject who have had a natural infection to EBOV and have been vaccinated again EBOV, comprising the use of at least one peptide chosen from the group consisting of the peptides listed in Tables 2, 3, 4 and 6 and combinations thereof and/or a collection or set of peptides shifted at least one residue across at least one peptide chosen from the group consisting of the peptides listed in Tables 2, 3, and 6 and combinations thereof.

A further embodiment is a method of identifying a subject who has been infected with EBOV and/or survived EBOV comprising the use of a peptide microarray comprising at least one peptide chosen from the group consisting of the peptides listed in Tables 2 and 3 and combinations thereof and/or a collection or set of peptides shifted at least one residue across at least one peptide chosen from the group consisting of the peptides listed in Tables 2 and 3 and combinations thereof.

A further embodiment is a method of identifying a subject who has been vaccinated against EBOV, comprising the use of a peptide microarray comprising at least one peptide chosen from the group consisting of the peptides listed in Tables 2, 4 and 6 and combinations thereof and/or a collection or set of peptides shifted at least one residue across at least one peptide chosen from the group consisting of the peptides listed in Tables 2, 4 and 6 and combinations thereof.

Yet a further embodiment is a method of identifying subject who have had a natural infection to EBOV and have been vaccinated again EBOV, comprising the use of a peptide microarray comprising a combination of peptides listed in Tables 2, 3, 4 and 6 and/or a collection or set of peptides shifted at least one residue across at least one peptide chosen from the group consisting of the peptides listed in Tables 2, 3, 4 and 6 and combinations thereof.

In some embodiments, the peptides listed in Table 6 are VSV10 and VSV11.

Also disclosed herein is a peptide with a sequence of the epitope core region of MGP3 TIGEWAFWETKKNLT (SEQ ID NO: 1) deriving from the immunoreactive epitopes of most interest MGP3-EIDTTIGEWAFWETKKNLTRKIR (SEQ ID NO: 4), MGP1-LILPQAKKDFFSSHPLREPVNATEDP (SEQ ID NO: 2) and MGP12-DKIDQIIHDFVDKTLPDQGDNDNWWTGWRQ (SEQ ID NO: 13), MGP3 with its immunoreactive core regions as TIGEWAFWETKKNLT (SEQ ID NO: 1) and MGP1 with LPQAKKDFFSSHPLRE (SEQ ID NO: 62). This epitope core region of MGP3 TIGEWAFWETKKNLT (SEQ ID NO: 1) can be used as a diagnostic marker and for virus discovery as well as for the development of immunogenic compositions.

In another aspect, the disclosure provides nucleic acids comprising a sequence encoding any of the disclosed peptides. In addition, the disclosure provides vectors comprising such nucleic acids, and host cells comprising such vectors. In certain embodiments, the vector is a shuttle vector. In other embodiments, the vector is an expression vector (e.g., a bacterial or eukaryotic expression vector). In certain embodiments, the host cell is a bacterial cell. In other embodiments, the host cell is a eukaryotic cell.

A platform comprising at least one peptide that is reactive with one or more primate antibodies associated with natural infection by EBOV, wherein the at least one peptide comprises an amino acid sequence in the group consisting of SEQ ID NOS: 2-17 and SEQ ID NOS: 18-36 or a portion thereof.

A platform comprising at least two peptides that are each reactive with one or more primate antibodies associated with vaccination against EBOV by a vesicular stomatitis virus-based vaccine, wherein a first peptide of the at least two peptides comprises an amino acid sequence in the group consisting of SEQ ID NOS: 2-17 or a portion thereof, and wherein a second peptide of the at least two peptides comprises an amino acid sequence in the group consisting of SEQ ID NOS: 37-45 and SEQ ID NOS: 58-61 or a portion thereof.

A platform comprising (a) at least two peptides that are each reactive with one or more primate antibodies associated with vaccination against EBOV by a vesicular stomatitis virus-based vaccine, and (b) at least one peptide that is reactive with one or more primate antibodies associated with natural infection by EBOV, wherein the at least two peptides of (a) comprise (i) a first peptide comprising an amino acid sequence in the group consisting of SEQ ID NOS: 2-17 or a portion thereof, and (ii) a second peptide comprises an amino acid sequence in the group consisting of SEQ ID NOS: 37-45 and SEQ ID NOS: 58-61 or a portion thereof, and wherein the at least one peptide of (b) comprises an amino acid sequence chosen from the group consisting of the sequences listed in SEQ ID NOS: 2-17 and the sequences listed in SEQ ID NOS: 18-36 or a portion thereof.

A platform comprising at least one peptide that is reactive with one or more primate antibodies associated with natural infection by a filovirus, wherein the at least one peptide comprises an amino acid sequence having SEQ ID NO: 1. In embodiments, the platform further comprises SEQ ID NO: 62.

A platform comprising at least one peptide that is reactive with one or more neutralizing primate antibodies associated with natural infection by EBOV, wherein the at least one peptide comprises an amino acid sequence having SEQ ID NO: 13.

A collection of isolated peptides for use in identifying primate antibodies associated with natural infection by EBOV, comprising at least one peptide comprises an amino acid sequence in the group consisting of SEQ ID NOS: 2-17 and SEQ ID NOS: 18-36 or a portion thereof.

A collection of isolated peptides comprising at least two peptides for use in identifying primate antibodies associated with vaccination against EBOV by a vesicular stomatitis virus-based vaccine wherein a first peptide of the at least two peptides comprises an amino acid sequence in the group consisting of SEQ ID NOS: 2-17 or a portion thereof, and wherein a second peptide of the at least two peptides comprises an amino acid sequence in the group consisting of SEQ ID NOS: 37-45 and SEQ ID NOS: 58-61 or a portion thereof.

A method of identifying a subject who has been infected with EBOV, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of an antibody to an epitope of a natural infection being present in the sample and identifies the subject as being previously infected with EBOV.

A method of identifying a subject who has been vaccinated against EBOV, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of an antibody to an epitope of vaccination to EBOV being present in the sample and identifies the subject as being previously vaccinated with EBOV.

A method of identifying a subject who has been infected with EBOV and/or vaccinated against EBOV, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of an antibody to an epitope of a natural infection and/or vaccination being present in the sample and identifies the subject as being previously infected with, and/or vaccinated against EBOV.

A method of identifying a subject who has been infected with a filovirus, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of an antibody to an epitope of a natural infection and/or vaccination being present in the sample and identifies the subject as being previously infected with a filovirus.

A method of identifying a subject as having a neutralizing antibody for EBOV, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of a neutralizing antibody for EBOV being present in the sample and identifies the subject as having a neutralizing antibody for EBOV.

A kit comprising a platform described herein and one or more detectably-labeled antibodies or fragments thereof which bind a primate antibody.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For the purpose of illustrating the invention, there are depicted in drawings certain embodiments of the invention. However, the invention is not limited to the precise arrangements and instrumentalities of the embodiments depicted in the drawings.

FIG. 1. Experimental plan for 12 Rhesus macaques vaccinated with VSV-EBOV-Makona-GP and 4 non-human primates (NHP) with VSV-MARV-Angola-GP, challenged with EBOV Makona.

FIG. 2. Immunoreactive epitopes in the GP.

FIG. 3. Immunoreactive epitopes in the sGP.

FIG. 4. Experimental plan for 10 humans vaccinated with VSV-EBOV-GP.

FIGS. 5A-5B. Graphs of ELISA of NHP samples with biotinylated peptide MGP3 (DTTIGEWAFWETKKNLTRK (SEQ ID NO: 63)) (FIG. 5A) and VSV5 (GKKSKKLGIAPPPYEEDTS (SEQ ID NO: 64)) (FIG. 5B) at dilution 1:100.

FIGS. 6A-6B. Graphs of ELISA of Human samples with of biotinylated peptide MGP3 (DTTIGEWAFWETKKNLTRK (SEQ ID NO: 63)) (FIG. 6A) and VSV5 (GKKSKKLGIAPPPYEEDTS (SEQ ID NO: 64)) (FIG. 6B) at dilution 1:100.

FIG. 7. Conserved Domain immunoreactive epitope in GP/sGP region of Filoviruses with diagnostic potential. Bombali (SEQ ID NO: 76), Ebola (SEQ ID NO: 77), Tai (SEQ ID NO: 78), Bundibugyo (SEQ ID NO: 79), Zaire (SEQ ID NO: 80), Sudan (SEQ ID NO: 81), and Reston (SEQ ID NO: 82) sequences are shown in the alignment.

FIG. 8. Crystal structure of monomer EBOV-GP.

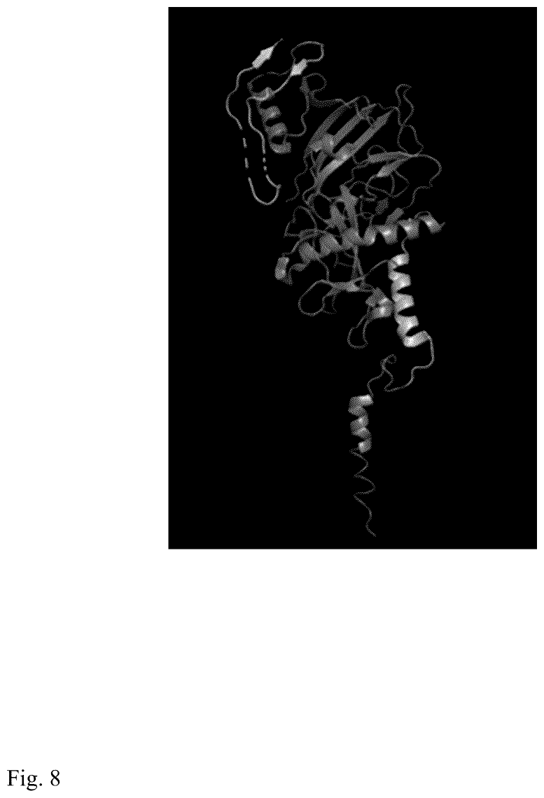

FIG. 9: Comparison of high-density peptide array data for selected epitopes (MV40E2, MNP1, MGP3, MGP7, VSV5) to low-density peptide array. Top row, left to right, SEQ ID NOS: 67-71, respectively, and bottom row, left to right, is SEQ ID NOS: 72-75.

### DETAILED DESCRIPTION OF THE DISCLOSURE

A platform comprising at least one peptide that is reactive with one or more primate antibodies associated with natural infection by EBOV, wherein the at least one peptide comprises an amino acid sequence in the group consisting of SEQ ID NOS: 2-17 and SEQ ID NOS: 18-36 or a portion thereof.

A platform comprising at least two peptides that are each reactive with one or more primate antibodies associated with vaccination against EBOV by a vesicular stomatitis virus-based vaccine, wherein a first peptide of the at least two peptides comprises an amino acid sequence in the group consisting of SEQ ID NOS: 2-17 or a portion thereof, and wherein a second peptide of the at least two peptides comprises an amino acid sequence in the group consisting of SEQ ID NOS: 37-45 and SEQ ID NOS: 58-61 or a portion thereof.

A platform comprising (a) at least two peptides that are each reactive with one or more primate antibodies associated with vaccination against EBOV by a vesicular stomatitis virus-based vaccine, and (b) at least one peptide that is reactive with one or more primate antibodies associated with natural infection by EBOV, wherein the at least two peptides of (a) comprise (i) a first peptide comprising an amino acid sequence in the group consisting of SEQ ID NOS: 2-17 or a portion thereof, and (ii) a second peptide comprises an amino acid sequence in the group consisting of SEQ ID NOS: 37-45 and SEQ ID NOS: 58-61 or a portion thereof, and wherein the at least one peptide of (b) comprises an amino acid sequence chosen from the group consisting of the sequences listed in SEQ ID NOS: 2-17 and the sequences listed in SEQ ID NOS: 18-36 or a portion thereof.

In embodiments, (a) and (b) are spatially separated on the platform for distinguishable identification.

In embodiments, the peptide sequences are chosen from MV40E2 SEQ ID NO: 32, MNP1 SEQ ID NO: 18; MGP3 SEQ ID NO: 48, MGP7 SEQ ID NO: 52, VSV5 SEQ ID NO: 59. In embodiments, the peptide sequences are SEQ ID NOS: 67-71.

A platform comprising at least one peptide that is reactive with one or more primate antibodies associated with natural infection by a filovirus, wherein the at least one peptide comprises an amino acid sequence having SEQ ID NO: 1. In embodiments, the platform further comprises SEQ ID NO: 62.

A platform comprising at least one peptide that is reactive with one or more neutralizing primate antibodies associated with natural infection by EBOV, wherein the at least one peptide comprises an amino acid sequence having SEQ ID NO: 13.

A collection of isolated peptides for use in identifying primate antibodies associated with natural infection by EBOV, comprising at least one peptide comprises an amino acid sequence in the group consisting of SEQ ID NOS: 2-17 and SEQ ID NOS: 18-36 or a portion thereof.

A collection of isolated peptides comprising at least two peptides for use in identifying primate antibodies associated with vaccination against EBOV by a vesicular stomatitis virus-based vaccine wherein a first peptide of the at least two peptides comprises an amino acid sequence in the group consisting of SEQ ID NOS: 2-17 or a portion thereof, and wherein a second peptide of the at least two peptides comprises an amino acid sequence in the group consisting of SEQ ID NOS: 37-45 and SEQ ID NOS: 58-61 or a portion thereof.

A method of identifying a subject who has been infected with EBOV, comprising:
a. contacting a sample from the subject with a platform described herein, under conditions sufficient to allow binding of antibody(ies) in the sample to peptide(s) of the platform so as to form an antibody-peptide complex; and
b. detecting any antibody-peptide complex so formed;
wherein detection of the complex is indicative of an antibody associated with a EBOV natural infection being present in the sample and identifies the subject as being previously infected with EBOV.

A method of identifying a subject who has been vaccinated against EBOV, comprising:
a. contacting a sample from the subject with a platform described herein, under conditions sufficient to allow binding of antibody(ies) in the sample to the first peptide, and to allow binding of antibody(ies) in the sample to the second peptide of the platform so as to form a first antibody-peptide complex and a second antibody-peptide complex, respectively; and
b. detecting any antibody-peptide complexes so formed;
wherein detection of both a first antibody-peptide complex and of a second antibody-peptide complex is indicative of antibodies associated with vaccination against EBOV with a vesicular stomatitis virus-based vaccine being present in the sample and identifies the subject as being previously vaccinated with an EBOV vesicular stomatitis virus-based vaccine.

A method of identifying a subject who has been infected with EBOV and/or vaccinated against EBOV, comprising:
a. contacting a sample from the subject with a platform described herein, under conditions sufficient to (a) allow binding of antibody(ies) in the sample to the first peptide comprising an amino acid sequence of at least five consecutive amino acids of a sequence in the group consisting of SEQ ID NOS: 2-17, and allow binding of antibody(ies) in the sample to the second peptide comprising an amino acid sequence of at least five consecutive amino acids of a sequence in the group consisting of SEQ ID NOS: 37-45 and SEQ ID NOS: 58-61, and also (b) allow binding of antibody(ies) in the sample to a third peptide comprising an amino acid sequence chosen from the group consisting of the sequences listed in SEQ ID NOS: 2-17 and SEQ ID NOS: 18-36, so as to permit formation of (a) a first antibody-first peptide complex and a second antibody-second peptide complex and/or (b) a third antibody-third peptide complex, respectively; and
b. detecting any antibody-peptide complexes so formed;
wherein detection of both a first antibody-first peptide complex and of a second antibody-second peptide complex is indicative of antibodies associated with vaccination against EBOV with a vesicular stomatitis virus-based vaccine being present in the sample and identifies the subject as being previously vaccinated with an EBOV vesicular stomatitis virus-based vaccine, and wherein detection of a third antibody-third peptide complex is indicative of antibodies associated with natural EBOV infection being present in the sample.

A method of identifying a subject who has been infected with a filovirus, comprising:
a. contacting a sample from the subject with the platform of claim 4, under conditions sufficient to allow binding of antibody(ies) to the peptide(s) thereof; and
b. detecting formation of an antibody-peptide complex comprising said one or more peptides;
wherein formation of the complex is indicative of an antibody associated with a filovirus, infection being present in the sample and identifies the subject as being previously infected with a filovirus.

A method of identifying a subject as having a neutralizing antibody for EBOV, comprising:
a. contacting a sample from the subject with the platform of claim 5, under conditions sufficient to allow binding of antibody(ies) to peptide(s) thereof; and
b. detecting formation of an antibody-peptide complex comprising said one or more peptides;
wherein formation of the complex is indicative of a neutralizing antibody for EBOV being present in the sample and identifies the subject as having a neutralizing antibody for EBOV.

A portion of the peptides listed by SEQ ID NO herein means a sequence of consecutive amino acids of said peptides, but less than the whole sequence set forth in a given SEQ ID NO. Portions can be 3 consecutive amino acids, be 4 consecutive amino acids, be 5 consecutive amino acids, be 6 consecutive amino acids, be 7 consecutive amino acids, 8 consecutive amino acids, be 9 consecutive amino acids, be 10 consecutive amino acids, be 11 consecutive amino acids, be 12 consecutive amino acids, 13 consecutive amino acids, be 14 consecutive amino acids, be 15 consecutive amino acids, be 16 consecutive amino acids, be 17 consecutive amino acids, be 18 consecutive amino acids, be 19 consecutive amino acids, be 20 consecutive amino acids, be 21 consecutive amino acids, be 22 consecutive amino acids, etc. of said peptides set forth in the SEQ ID NOS. herein. In embodiments, the portions are 12 consecutive amino acids of a peptide described in Tables 2-6 herein. In embodiments, the portions are 16 consecutive amino acids of a peptide described in Tables 2-6 herein. In embodiments, the portions are one or two amino acids less than the full length peptides described in Tables 2-6 herein.

Peptides of 12 amino acids were constructed based upon work that shows that antibodies bind to linear peptide sequences ranging from 5 to 9 amino acids in length and bind most efficiently when targets are flanked by additional amino acids (Buus et al. 2012). However, peptides containing less than 12 amino acids in length and more than 12 amino acids in length can be used. Peptides 13 amino acids in length, 14 amino acids in length, 15 amino acids in length, 16 amino acids in length, 17 amino acids in length, 18 amino acids in length, 19 amino acids in length, 20 amino acids in length, up to 25 amino acids in length, up to 30 amino acids in length, up to 35 amino acids in length, up to 40 amino acids in length, and up to 50 amino acids in length can be used.

A method of identifying a subject who has been infected with EBOV, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of an antibody to an epitope of a natural infection being present in the sample and identifies the subject as being previously infected with EBOV.

A method of identifying a subject who has been vaccinated against EBOV, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of an antibody to an epitope of vaccination to EBOV being present in the sample and identifies the subject as being previously vaccinated with EBOV.

A method of identifying a subject who has been infected with EBOV and/or vaccinated against EBOV, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of an antibody to an epitope of a natural infection and/or vaccination being present in the sample and identifies the subject as being previously infected with, and/or vaccinated against EBOV.

A method of identifying a subject who has been infected with a filovirus, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of an antibody to an epitope of a natural infection and/or vaccination being present in the sample and identifies the subject as being previously infected with a filovirus.

A method of identifying a subject as having a neutralizing antibody for EBOV, comprising:
a. contacting a sample from the subject with a platform described herein, under conditions sufficient to allow binding of antibody(ies) to the peptide(s) thereof; and
b. detecting formation of an antibody-peptide complex comprising said one or more peptides;
wherein formation of the complex is indicative of a neutralizing antibody for EBOV being present in the sample and identifies the subject as having a neutralizing antibody for EBOV.

A kit comprising a platform described herein and one or more detectably-labeled antibodies or fragments thereof which bind a primate antibody.

In embodiments, the detectably-labeled antibodies or fragments thereof are labeled with an enzyme. In embodiments, the enzyme is horseradish peroxidase (HRP) or alkaline phosphatase (AP).

In embodiments, the one or more detectably-labeled antibodies or fragments thereof bind human a IgG antibody or a IgM antibody.

In embodiments, the one or more detectably-labeled antibodies or fragments thereof bind human a Fab of an IgG antibody.

In embodiments, the kit further comprises a buffer solution, a wash solution, and/or an enzyme substrate or chromogen.

In embodiments, the platform comprises a solid phase or semi-solid phase to which the peptides are attached, adsorbed, immobilized, bound or enclosed fully or partially. In embodiments, the platform comprises an ELISA plate or ELISA plate coating, a microarray, a lateral flow device, a Western blot membrane or gel, a radioimmunoassay, or a bead of a bead-based assay.

In embodiments, the solid phase comprises a plastic polymer or a glass.

In embodiments, the primate is a human.

The present invention provides high-density peptide microarray to identify immunogenic epitopes from proteomes of filoviruses.

The present invention also provides identification of peptides that enable specific and sensitive serological detection of adaptive immune responses to a wide range of clinically important high threat pathogens circulating in sub-Saharan Africa on a wide range of platforms including but not limited to phage display, microarray, ELISA, RIA, lateral flow, western blot, and bead-based assays.

Immunoassays are usually used to measure cell surface antigens. Typically, immunofluorescence using flow cytometry is the immunoassay of choice. However, other immunoassays may be used, for example enzyme linked immunosorbant assays (ELISA). This technique is based upon the special properties of antigen-antibody interactions with simple phase separations to produce powerful assays for detecting biological molecules.

One well-known and highly specific ELISA is a sandwich ELISA. In this assay, the antibody is bound to the solid phase or support, which is then contacted with the sample being tested to extract the antigen from the sample by formation of a binary solid phase antibody:antigen complex. After a suitable incubation period, the solid support is washed to remove the residue of the fluid sample and then contacted with a solution containing a known quantity of labeled antibody.

In specific embodiments, the antibody:antigen complex is measured in a patient that has undergone at least one course, e.g., an injection, of immunotherapy with a therapeutic antibody.

Typically, sandwich immunoassays are performed on devices using a capture antibody immobilized on a filter or membrane, either directly or indirectly through trapping of antibody-coated latex particles. The specimen or an extract of it is applied to a device containing the filter or membrane with immobilized capture antibody. These commercial devices utilize capillary action to cause the sample and reagents to flow through the filter or membrane into an absorbent material lying beneath it. A sandwich is eventually formed in which one member is labeled with an appropriate signal-generating substituent, e.g. an enzyme label. Addition of substrate will detect the enzyme label by reacting with the enzyme to produce a colored reaction product. Since the enzyme is attached to a member of a sandwich whose existence requires the presence of the antigen, the colored reaction indicates the presence of the antigen as well.

U.S. Pat. No. 4,366,241, issued to Tom et al., on Dec. 28, 1982, which discloses a method and apparatus for performing sandwich immunoassays. The apparatus is described as comprising an immunosorbing layer to which a member of an immunological pair is attached, a liquid absorbing member and a bibulous liquid flow-resistant disk. The bibulous liquid flow-resistant disk retards entry of liquid to the absorbent layer so that a uniform flow rate is achieved for liquid flowing through the immunosorbing member. Flow is not restricted to the particular area where the member of an immunological pair is immobilized on the immunosorbing member.

Tom et al. further discloses preventing signal production in a first layer of a multilayer immunosorbing zone by adding an enzyme to an intermediate layer to prevent the transfer of compounds from the detection layer. The example provided in column 13 states that the detector enzyme is present in a first layer and in a third layer. The system is designed so that detection occurs in the third layer. The substrate is not added to the first layer; instead it is generated in situ in the third layer. Another enzyme is present in the layer between the first layer and third layer which prevents any substrate from migrating from the third layer to the first layer so that detection occurs only in the third layer. There is no teaching of reducing background color in an absorbent layer by treating the absorbent layer with an enzyme inhibitor which acts directly on the enzyme.

U.S. Pat. No. 4,407,943, issued to Cole et al. on Oct. 4, 1983, discloses immobilization of an antigen or antibody on zein-coated internal and external surfaces of a microporous membrane whose surfaces are rendered immunochemically reactive. It is disclosed that structures having large ratios of surface exposed to the volume of fluid flowing through the membrane are used in an attempt to increase the probability of reactive contact with the surface and reduce reaction time. The incubation time is governed by a technique whereby fluid is applied dropwise to the upper surface of the membrane so that the rate of flow of fluid is governed only by the hydrostatic pressure exerted by the drop as it rests on top of the membrane. An absorbent layer is not employed to exert a capillary action to pull the reagents therethrough.

U.S. Pat. No. 4,246,339, issued to Cole et al. on Jan. 20, 1981, discloses a multilayered device for testing liquid samples for the presence of predetermined components. The test device comprises telescoping top and base members which define a liquid reservoir, the top member has one or more test wells which open to the periphery of a microporous membrane. The microporous membrane has a co-reactant immobilized to its internal and external surfaces, which co-reactant is capable of reacting with the analyte. When a resilient means is depressed, the membrane contacts an absorbent member capable, by blotting or capillary action, of absorbing all the liquid passed through the test wells. Unless there is an absorbent layer and a means for bringing it into contact with the lower surface of the membranes, liquid will not pass through the membranes. When the device is not depressed no liquid will pass through the microporous membrane. The absorbent layer comprises a surface layer which is substantially non-wettable, i.e., for normal aqueous solutions it is comparable to the inner lining of diapers, and a substantially wettable layer. The high-density peptide microarray can also be used with lateral flow assay test kits. They are currently available for testing for a wide variety of medical and environmental conditions or compounds, such as a hormone, a metabolite, a toxin, or a pathogen-derived antigen.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein, the term “sample” means any substance containing or presumed to contain antibodies. The sample can be of natural or synthetic origin and can be obtained by any means known to those of skill in the art. The sample can be a sample of tissue or fluid isolated from a subject including but not limited to, plasma, serum, whole blood, spinal fluid, semen, amniotic fluid, lymph fluid, synovial fluid, urine, tears, blood cells, organs, and tissue. Samples can be research, clinical, or environmental. Sample can also be blood products used to transfuse or treat. Samples can also be synthetic and include but are not limited to in vitro cell culture constituents including but not limited to conditioned medium, recombinant cells, and cell components.

As used herein, the term “subject” means any organism including, without limitation, a mammal such as a mouse, a rat, a dog, a guinea pig, a ferret, a rabbit and a primate. In the preferred embodiment, the subject is a human being, a pet or livestock animal.

The term “patient” as used in this application means a human subject.

The term “detection”, “detect”, “detecting” and the like as used herein means as used herein means to discover the presence or existence of.

The terms “identification”, “identify”, “identifying” and the like as used herein means to recognize exposure to a specific virus or viruses in sample from a subject.

The term “peptide” includes any sequence of two or more amino acids. Peptide sequences specifically recited herein are written with the amino terminus on the left and the carboxy terminus on the right.

The term “amino acid,” includes the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate, hippuric acid, octahydroindole-2-carboxylic acid, statine, 1,2,3,4,-tetrahydroisoquinoline-3-carboxylic acid, penicillamine, omithine, citruline, alpha-methylalanine, para-benzoylphenylalanine, phenylglycine, propargylglycine, sarcosine, and tert-butylglycine). The term also includes natural and unnatural amino acids bearing a conventional amino protecting group (e.g. acetyl or benzyloxycarbonyl), as well as natural and unnatural amino acids protected at the carboxy terminus (e.g. as a (C1-C6) alkyl, phenyl or benzyl ester or amide).

An “antigen” (from antibody-generating) or “immunogen” is a substance that prompts the generation of antibodies and can cause an immune response. They may also be used for diagnostic or patient selection or characterization purposes.

Antibodies (also known as immunoglobulins (Ig)) are globulin proteins that are found in blood or other bodily fluids of vertebrates and are used by the immune system to identify and neutralize foreign objects, such as bacteria and viruses. They are typically made of basic structural units—each with two large heavy chains and two small light chains—to form, for example, monomers with one unit, dimers with two units or pentamers with five units. Antibodies are produced by B cells. There are several different types of antibody heavy chains, and several different kinds of antibodies, which are grouped into different isotypes based on which heavy chain they possess. Five different antibody isotypes are known in mammals, which perform different roles, and help direct the appropriate immune response for each different type of foreign object they encounter.

Although the general structure of all antibodies is very similar, a small region at the tip of the protein is extremely variable, allowing millions of antibodies with slightly different tip structures to exist. This region is known as the hypervariable region. Each of these variants can bind to a different target, known as an antigen. This huge diversity of antibodies allows the immune system to recognize an equally wide diversity of antigens. The part of the antigen recognized by an antibody is termed an “epitope.” These epitopes bind with their antibody in a highly specific interaction, called induced fit, which allows antibodies to identify and bind only their specific epitope in the matching antigen(s) in the midst of the millions of different molecules that make up an organism. Recognition of an antigen by an antibody tags it for attack by other parts of the immune system. Antibodies can also neutralize targets directly by, for example, binding to a part of a pathogen that it needs to cause an infection. Production of antibodies is the main function of the humoral immune system.

As used herein, the term “isolated” and the like means that the referenced material is free of components found in the natural environment in which the material is normally found. In particular, isolated biological material is free of cellular components. An isolated peptide or protein may be associated with other proteins or nucleic acids, or both, with which it associates in the cell, or with cellular membranes if it is a membrane-associated protein. An isolated material may be, but need not be, purified.

The term “substantially purified,” as used herein, refers to a molecule, such as a peptide, that is substantially free of cellular material (proteins, lipids, carbohydrates, nucleic acids), culture medium, chemical precursors, chemicals used in synthesis of the peptide, or combinations thereof. A peptide that is substantially purified has less than about 40%, 30%, 25%, 20%, 15%, 10%, 5%, 2%, 1% or less of the cellular material, culture medium, other polypeptides, chemical precursors, and/or chemicals used in synthesis of the peptide. Accordingly, a substantially pure molecule, such as a peptide, can be at least about 60%, 70%, 75%, 80%, 85%, 90%, 95%, 98%, or 99%, by dry weight, the molecule of interest.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The current disclosure enhances the differential diagnosis by establishing a new serologic assay platform for profiling a subject's pathogen exposure history, as well as providing peptides which distinguish whether a subject has had a natural infection to EBOV, been vaccinated against EBOV or both.

In contrast to molecular diagnostics where advances in technology such as polymerase chain reaction and high-throughput screening have dramatically improved sensitivity, specificity and breadth over the past 20 years, serologic methods remained largely unchanged. This lag is important given the role of serology in establishing the distribution and frequency of infection, testing the significance of association between the finding of an agent and disease, and in focusing efforts in pathogen discovery. Described herein is a sensitive, unbiased, highly multiplexed platform for diagnostic serology. This peptide array-based platform will enable new strategies for investigating the epidemiology and pathogenesis of acute and chronic diseases due to infection and for monitoring humoral responses to vaccines and immunomodulatory drugs. It will also serve as a screening tool for rapid selection of key informative peptides that can be used in established, inexpensive, alternative platforms including lateral flow immunoassays. Such applications will have practical utility for both clinical medicine and public health by enabling retrospective differential diagnosis of an infectious illness (when genetic footprints of the agent may no longer be present), and in facilitating outbreak investigation and surveillance.

Serodiagnosis of infection by several pathogens circulating in sub-Saharan Africa can be impeded by immunological cross-reactivity among them. Disclosed herein are peptides that enable discrimination between exposure to the various pathogens.

Disclosed herein is a sensitive, highly multiplexed, microarray-based assay that enables the discrimination of antibody responses to linear epitopes specific to EBOV vaccination, natural infection of EBOV, filoviruses, Lyssa viruses, Dengue viruses (DENV), yellow fever virus (YFV), West Nile virus (WNV), Selmiki forest virus, Rift valley fever virus (RVF), Measles virus, Mumps virus, Rubella virus, and selective immunogenic proteins of Mycobacterium tuberculosis, Salmonella typhi, and Plasmodium falciparum.

A high-density (390,894 feature) peptide microarray was used to identify immunoreactive peptides for differential serodiagnosis of exposure to a broad range microbial agents including filoviruses. Immunoreactive peptides were identified using (Rhesus macaque) plasma collected after Recombinant vesicular stomatitis virus-Zaire Ebola virus (VSV-EBOV-GP) vaccination and challenge with EBOV Makona strain, and plasma samples from humans immunized with VSV-EBOV-GP vaccine. Patterns of immunoreactivity correlated with infection with distinct strains of Ebola, other filoviruses, and the vesicular stomatitis virus vaccine vector (VSIV). The findings indicate that specific peptides can be used in immunoassays to discriminate between post-immunization immunity from post infection immunity.

Additionally a peptide was identified in conserved domain of GP of filoviridae that could be useful to discover or study zoonosis and reverse-zoonosis events of known and yet to discovered filoviruses.

### Isolated Peptides Specific and Sensitive for Antibodies (Reactive Peptides)

The present disclosure includes isolated peptides which are strongly reactive with, and sensitive to antibodies EBOV vaccination, natural infection of EBOV, filoviruses in a patient sample. These peptides can be used in any type of serological assay or platform, now known or later developed, to screen for the presence of antibodies. These peptides can also be used to test for and monitor humoral responses to vaccines and immunomodulatory drugs, thus, being useful for the development of treatment and preventative agents.

Thus, one embodiment of the current disclosure is an isolated peptide which is reactive with, and specific for one or more filovirus pathogens in Table 1 or EBOV vaccine. A further embodiment comprises a collection or set of peptides comprising amino acid sequences shifted at least one residue across any of the peptides which is reactive with, and specific for one or more filovirus pathogens in Table 1 or EBOV vaccine.

Also disclosed herein is the identification of EBOV peptides which can identify and differentiate subjects who have been infected with EBOV, those who have been vaccinated against EBOV, those who are survivors of EBOV, and those who received a vaccination and had a past natural infection. These include:

Sixteen EBOV peptides (MGP1-MSGP4) listed in Table 2 and 19 EBOV peptides (MNP1-ML1) listed in Table 3 can be used to identify people who have been infected with EBOV.

Sixteen EBOV peptides (MGP1-MSGP4) listed in Table 2, in combination with 9 VSV peptides (VSV1-VSV9) listed in Table 4 and 2 additional VSV peptides (VSV10 and VSV11) listed in Table 6 can be used to identify people who have been Vaccinated.

Sixteen EBOV peptides (MGP1-MSGP4) listed in Table 2 and 19 EBOV peptides (MNP1-ML1) listed in Table 3 can also identify the EBOV survivors.

Combination of 16 EBOV peptides (MGP1-MSGP4) listed in Table 2, 19 EBOV peptides (MNP1-ML1) listed in Table 3, 9 VSV peptides (VSV1-VSV9) listed in Table 4, and 2 additional VSV peptides (VSV10 and VSV11) can be used to determine individuals who received vaccination and had a past natural infection both.

Thus, a further embodiment of the current disclosure is an isolated peptide chosen from the group consisting of the peptides listed in Tables 2, 3, 4 and 6. A further embodiment of the present disclosure are collections or sets of peptides comprising amino acid sequences shifted at least one residue across one or more peptides chosen from the group consisting of the peptides chosen from the group consisting of the peptides listed in Tables 2, 3, 4 and 6 and combinations thereof.

A further embodiment of the current disclose is a peptide with a sequence of the epitope core region of MGP3 TIGEWAFWETKKNLT (SEQ ID NO: 1) which can be used as a diagnostic marker and for virus discovery as well as for the development of immunogenic compositions.

The collections or sets can contain the shifted peptides that are 6 amino acids in length, 7 amino acids in length, 8 amino acids in length, 9 amino acids in length, 10 amino acids in length, 11 amino acids in length, and up to 12 amino acids in length.

In one embodiment, each peptide in the set or collection is 12 amino acids in length.

Peptides of 12 amino acids were constructed based upon work that shows that antibodies bind to linear peptide sequences ranging from 5 to 9 amino acids in length and bind most efficiently when targets are flanked by additional amino acids (Buus et al. 2012). However, peptides containing less than 12 amino acids in length and more than 12 amino acids in length can be used. Peptides 13 amino acids in length, 14 amino acids in length, 15 amino acids in length, 16 amino acids in length, 17 amino acids in length, 18 amino acids in length, 19 amino acids in length, 20 amino acids in length, up to 25 amino acids in length, up to 30 amino acids in length, up to 35 amino acids in length, up to 40 amino acids in length, and up to 50 amino acids in length can be used.

The number of peptides in a collection or set can range from 2 peptides to a number in the thousands to tens of thousands to hundreds of thousands.

In another aspect, the disclosure provides compositions comprising two or more disclosed peptides.

In certain embodiments, the peptides described herein are produced by synthetic chemistry (i.e., a “synthetic peptide”). In other embodiments, peptides of the invention are produced biologically. An isolated peptide of the invention can be in water, a buffer, or in a dry form awaiting reconstitution, e.g., as part of a kit. An isolated peptide of the present invention can be in the form of a pharmaceutically acceptable salt. Suitable acids and bases that are capable of forming salts with the peptides of the present invention are well known to those of skill in the art and include inorganic and organic acids and bases.

In certain embodiments, the peptides described herein are modified. The peptides may be modified by a variety of techniques, such as by denaturation with heat and/or a detergent (e.g., SDS). Alternatively, peptides may be modified by association with one or more further moieties. The association can be covalent or non-covalent, and can be, for example, via a terminal amino acid linker, such as lysine or cysteine, a chemical coupling agent, or a peptide bond. The additional moiety can be, for example, a ligand, a ligand receptor, a fusion partner, a detectable label, an enzyme, or a substrate that immobilizes the peptide.

The disclosed peptides can be conjugated to a ligand, such as biotin (e.g., via a cysteine or lysine residue), a lipid molecule (e.g., via a cysteine residue), or a carrier protein (e.g., serum albumin, immunoglobulin Fc domain, keyhole limpet hemocyanin (KLH) via e.g., a cysteine or lysine residue). Attachment to ligands, such as biotin, can be useful for associating the peptide with ligand receptors, such as avidin, streptavidin, polymeric streptavidin, or neutravidin. Avidin, streptavidin, polymeric streptavidin, or neutravidin, in turn, can be linked to a signaling moiety (e.g., an enzyme, such as horse radish peroxidase (HRP) or alkaline phosphatase (ALP), or other moiety that can be visualized, such as a metallic nanoparticle or nanoshell (e.g., colloidal gold) or a fluorescent moiety), or a solid substrate (e.g., nitrocellulose membrane). Alternatively, the peptides of the invention can be fused or linked to a ligand receptor, such as avidin, streptavidin, polymeric streptavidin, or neutravidin, thereby facilitating the association of the peptides with the corresponding ligand, such as biotin and any moiety (e.g., signaling moiety) or solid substrate attached thereto. Examples of other ligand-receptor pairs are well-known in the art and can similarly be used.

The peptides can be fused to a fusion partner (e.g., a peptide or other moiety) that can be used to improve purification, to enhance expression of the peptide in a host cell, to aid in detection, and to stabilize the peptide. Examples of suitable compounds for fusion partners include carrier proteins (e.g., serum albumin, immunoglobulin Fc domain, KLH), and enzymes (e.g., horse radish peroxidase (HRP), beta-galactosidase, glutathione-S-transferase, alkaline phosphatase). The fusion can be achieved by means of a peptide bond. For example, peptides of the invention and fusion partners can be fusion proteins and can be directly fused in-frame or can comprise a peptide linker.

In addition, the disclosed peptides may be modified to include any of a variety of known chemical groups or molecules. Such modifications include, but are not limited to, glycosylation, acetylation, acylation, ADP-ribosylation, amidation, covalent attachment to polyethylene glycol (e.g., PEGylation), covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, ubiquitination, modifications with fatty acids, and transfer-RNA mediated addition of amino acids to proteins such as arginylation. Analogues of an amino acid (including unnatural amino acids) and peptides with substituted linkages are also included.

The disclosed peptides that consist of any of the sequences discussed herein may be modified by any of the discussed modifications. Such peptides still “comprise” or “consist of” the amino acids.

Modifications as set forth above are well-known to those of skill in the art and have been described in great detail in the scientific literature.

### Nucleic Acids Comprising a Sequence Encoding the Reactive Peptides

In another aspect, the disclosure provides nucleic acids comprising a sequence encoding any disclosed peptide. Nucleic acids of the invention can be single- or double-stranded. A nucleic acid can be RNA, DNA, cDNA, genomic DNA, chemically synthesized RNA or DNA or combinations thereof. The nucleic acids can be purified free of other components, such as proteins, lipids and other polynucleotides. For example, the nucleic acids can be 50%, 75%, 90%, 95%, 96%, 97%, 98%, 99%, or 100% purified. The nucleic acids encode the peptides described herein. Nucleic acids can comprise other nucleotide sequences, such as sequences coding for linkers, signal sequences, TMR stop transfer sequences, transmembrane domains, or ligands useful in protein purification such as glutathione-S-transferase, histidine tag, and staphylococcal protein A.

Methods for preparing polynucleotides operably linked to an expression control sequence and expressing them in a host cell are well-known in the art. See, e.g., U.S. Pat. No. 4,366,246. A nucleic acid of the invention is operably linked when it is positioned adjacent to or close to one or more expression control elements, which direct transcription and/or translation of the polynucleotide.

Thus, for example, the peptides described herein can be produced recombinantly following conventional genetic engineering techniques. To produce a recombinant peptide, a nucleic acid encoding the peptide is inserted into a suitable expression system. Generally, a recombinant molecule or vector is constructed in which the polynucleotide sequence encoding the selected peptide is operably linked to an expression control sequence permitting expression of the peptide. Numerous types of appropriate expression vectors are known in the art, including, e.g., vectors containing bacterial, viral, yeast, fungal, insect or mammalian expression systems. Methods for obtaining and using such expression vectors are well-known. For guidance in this and other molecular biology techniques used for compositions or methods of the invention, see, e.g., Sambrook et al., Molecular Cloning, A Laboratory Manual, current edition, Cold Spring Harbor Laboratory, New York; Miller et al, Genetic Engineering, 8:277-298 (Plenum Press, current edition), Wu et al., Methods in Gene Biotechnology (CRC Press, New York, N.Y., current edition), Recombinant Gene Expression Protocols, in Methods in Molecular Biology, Vol. 62, (Tuan, ed., Humana Press, Totowa, N.J., current edition), and Current Protocols in Molecular Biology, (Ausabel et al., Eds.) John Wiley & Sons, NY (current edition), and references cited therein.

Accordingly, the disclosure also provides vectors comprising nucleic acids described herein, and host cells comprising such vectors. In certain embodiments, the vector is a shuttle vector. In other embodiments, the vector is an expression vector (e.g., a bacterial or eukaryotic expression vector). In certain embodiments, the host cell is a bacterial cell. In other embodiments, the host cell is a eukaryotic cell.

### Platforms, Assays and Device for Using the Reactive Peptides

There are a number of different conventional assays for detecting formation of an antibody-peptide complex comprising a peptide or peptides of the invention. For example, the detecting step can comprise performing a microarray assay, an ELISA assay, performing an immunofluorescence assay, performing a lateral flow immunoassay, performing an agglutination assay, performing a wavelength shift assay, performing a Western blot, slot blot, or dot blot, analyzing the sample in an analytical or centrifugal rotor, or analyzing the sample with an electrochemical, optical, or opto-electronic sensor. These different assays are described herein and/or are well-known to those skilled in the art.

Thus, the peptides disclosed herein can be used in any assay, format or platform for antibody detection including but not limited to microarrays, ELISA, RIA, lateral flow, western blot, and bead-based assays, as well as those platforms that are later developed.

In certain embodiments, the assay comprises: immobilizing the antibody(s) in the sample; adding a peptide disclosed herein; and detecting the degree of antibody bound to the peptide, e.g., by the peptide being labeled or by adding a labeled substance, such as a labeled binding partner (e.g., streptavidin-HRP or streptavidin-colloidal gold complex) or a labeled antibody which specifically recognizes the peptide.

In other embodiments, the assay comprises: immobilizing a peptide disclosed herein; adding the sample containing antibodies; and detecting the amount of antibody bound to the peptide, e.g., by adding another peptide disclosed herein conjugated, directly or indirectly, to a label (e.g., metallic nanoparticle or metallic nanoshell, fluorescent label, or enzyme (e.g., horseradish peroxidase or alkaline phosphatase)) or by adding a labeled substance, such as a binding partner or a labeled antibody which specifically recognizes the sample antibodies (e.g., anti-human IgG antibodies, or anti-human IgM antibodies).

In other embodiments, the assay comprises: immobilizing a peptide disclosed herein; adding the sample containing antibodies; and detecting the amount of antibody bound to the peptide, e.g., by adding a first binding partner which specifically recognizes the sample antibodies (e.g., anti-human IgG antibodies, or anti-human IgM antibodies), and further adding a second binding partner, wherein the second binding partner is labeled and recognizes said first binding partner.

In still other embodiments, the assay comprises: reacting the peptide and the sample containing antibodies without any of the reactants being immobilized, and then detecting the amount of complexes of antibody and peptide, e.g., by the peptide being labeled or by adding a labeled substance, such as a labeled binding partner (e.g., streptavidin-HRP or streptavidin-colloidal gold complex) or a labeled antibody which specifically recognizes the peptide.

Immobilization of a peptide can be either covalent or non-covalent, and the non-covalent immobilization can be non-specific (e.g., non-specific binding to a polystyrene surface in a microtiter well). Specific or semi-specific binding to a solid or semi-solid carrier, support or surface, can be achieved by the peptide having, associated with it, a moiety which enables its covalent or non-covalent binding to the solid or semi-solid carrier, support or surface. For example, the moiety can have affinity to a component attached to the carrier, support or surface. In this case, the moiety may be, for example, a biotin or biotinyl group or an analogue thereof bound to an amino acid group of the peptide, and the component is then avidin, streptavidin, neutravidin, or an analogue thereof.

Suitable carriers, supports, and surfaces include, but are not limited to, metallic nanolayers, beads (e.g., magnetic beads, colloidal particles or metallic nanoparticles or nanoshells, such as colloidal gold, or particles or nanoparticles comprising silica, latex, polystyrene, polycarbonate, or PDVF), latex of co-polymers such as styrene-divinyl benzene, hydroxylated styrene-divinyl benzene, polystyrene, carboxylated polystyrene, beads of carbon black, non-activated or polystyrene or polyvinyl chloride activated glass, epoxy-activated porous magnetic glass, gelatin or polysaccharide particles or other protein particles, red blood cells, mono- or polyclonal antibodies or Fab fragments of such antibodies.

The protocols for immunoassays using antigens for detection of specific antibodies are well known in art. For example, a conventional sandwich assay can be used, or a conventional competitive assay format can be used.

Devices for performing specific binding assays, especially immunoassays, are known and can be readily adapted for use in the present methods. Solid-phase assay devices include microtiter plates, flow-through assay devices (e.g., lateral flow immunoassay devices), dipsticks, and immunocapillary or immunochromatographic immunoassay devices.

In embodiments, the solid or semi-solid surface or carrier is the floor or wall in a microtiter well, a filter surface or membrane (e.g., a nitrocellulose membrane or a PVDF (polyvinylidene fluoride) membrane), a hollow fiber, a beaded chromatographic medium (e.g., an agarose or polyacrylamide gel), a magnetic bead, a fibrous cellulose matrix, an HPLC matrix, an FPLC matrix, a substance having molecules of such a size that the molecules with the peptide bound thereto, when dissolved or dispersed in a liquid phase, can be retained by means of a filter, a substance capable of forming micelles or participating in the formation of micelles allowing a liquid phase to be changed or exchanged without entraining the micelles, a water-soluble polymer, or any other suitable carrier, support or surface.

In some embodiments, the peptide is provided with a suitable label which enables detection. Conventional labels may be used which are capable, alone or in concert with other compositions or compounds, of providing a detectable signal. Suitable labels include, but are not limited to, enzymes (e.g., HRP, beta-galactosidase, or alkaline phosphatase), fluorescent labels, radioactive labels, colored latex particles, and metal-conjugated labels (e.g., metallic nanolayers, metallic nanoparticle- or metallic nanoshell-conjugated labels). Suitable metallic nanoparticle or metallic nanoshell labels include, but are not limited to, gold particles, silver particles, copper particles, platinum particles, cadmium particles, composite particles, gold hollow spheres, gold-coated silica nanoshells, and silica-coated gold shells. Metallic nanolayers suitable for detectable layers include nanolayers comprised of cadmium, zinc, mercury, and noble metals, such as gold, silver, copper, and platinum.

Suitable detection methods include, but are not limited to, detection of an agent which is tagged, directly or indirectly, with a colorimetric assay (e.g., for detection of HRP or beta-galactosidase activity), visual inspection using light microscopy, immunofluorescence microscopy, including confocal microscopy, or by flow cytometry (FACS), autoradiography (e.g., for detection of a radioactively labeled agent), electron microscopy, immunostaining, subcellular fractionation, or the like. In one embodiment, a radioactive element (e.g., a radioactive amino acid) is incorporated directly into a peptide chain. In another embodiment, a fluorescent label is associated with a peptide via biotin/avidin interaction, association with a fluorescein conjugated antibody, or the like. In one embodiment, a detectable specific binding partner for the antibody is added to the mixture. For example, the binding partner can be a detectable secondary antibody or other binding agent (e.g., protein A, protein G, protein L or combinations thereof) which binds to the first antibody. This secondary antibody or other binding agent can be labeled with, for example, a radioactive, enzymatic, fluorescent, luminescent, metallic nanoparticle or metallic nanoshell (e.g. colloidal gold), or other detectable label, such as an avidin/biotin system. In another embodiment, the binding partner is a peptide disclosed herein, which can be conjugated directly or indirectly to an enzyme, such as horseradish peroxidase or alkaline phosphatase or other signaling moiety. In such embodiments, the detectable signal is produced by adding a substrate of the enzyme that produces a detectable signal, such as a chromogenic, fluorogenic, or chemiluminescent substrate.

In some embodiments, the detection procedure comprises visibly inspecting the antibody-peptide complex for a color change or inspecting the antibody-peptide complex for a physical-chemical change. Physical-chemical changes may occur with oxidation reactions or other chemical reactions. They may be detected by eye, using a spectrophotometer, or the like.

One assay format is a lateral flow immunoassay format. Antibodies to human or animal immunoglobulins can be labeled with a signal generator or reporter (e.g., colloidal gold) that is dried and placed on a glass fiber pad (sample application pad or conjugate pad). The diagnostic peptide is immobilized on membrane, such as nitrocellulose or a PVDF (polyvinylidene fluoride) membrane. When a sample is applied to the sample application pad (or flows through the conjugate pad), it dissolves the labeled reporter, which then binds to all antibodies in the sample. The resulting complexes are then transported into the next membrane (PVDF or nitrocellulose containing the diagnostic peptide) by capillary action. If antibodies against the diagnostic peptide are present, they bind to the diagnostic peptide striped on the membrane, thereby generating a signal (e.g., a band that can be seen or visualized). An additional antibody specific to the labeled antibody or a second labeled antibody can be used to produce a control signal.

An alternative format for the lateral flow immunoassay comprises the peptides or compositions being conjugated to a ligand (e.g., biotin) and complexed with labeled ligand receptor (e.g., streptavidin-colloidal gold). The labeled peptide complexes can be placed on the sample application pad or conjugate pad. Anti-human IgG/IgM or anti-animal IgG/IgM antibodies are immobilized on a membrane, such as nitrocellulose of PVDF, at a test site. When sample is added to the sample application pad, antibodies in the sample react with the labeled peptide complexes such that antibodies that bind to peptides become indirectly labeled. The antibodies in the sample are then transported into the next membrane (PVDF or nitrocellulose containing the diagnostic peptide) by capillary action and bind to the immobilized anti-human IgG/IgM or anti-animal IgG/IgM antibodies. If any of the sample antibodies are bound to the labeled peptides, the label associated with the peptides can be seen or visualized at the test site.

Another assay for the screening of blood products or other physiological or biological fluids is an enzyme linked immunosorbent assay, i.e., an ELISA. Typically, in an ELISA, isolated peptides or collection or set of peptides disclosed herein, are adsorbed to the surface of a microtiter well directly or through a capture matrix (e.g., an antibody). Residual, non-specific protein-binding sites on the surface are then blocked with an appropriate agent, such as bovine serum albumin (BSA), heat-inactivated normal goat serum (NGS), or BLOTTO (a buffered solution of nonfat dry milk which also contains a preservative, salts, and an antifoaming agent). The well is then incubated with a biological sample suspected of containing specific antibody. The sample can be applied neat, or more often it can be diluted, usually in a buffered solution which contains a small amount (0.1-5.0% by weight) of protein, such as BSA, NGS, or BLOTTO. After incubating for a sufficient length of time to allow specific binding to occur, the well is washed to remove unbound protein and then incubated with an optimal concentration of an appropriate anti-immunoglobulin antibody that is conjugated to an enzyme or other label by standard procedures and is dissolved in blocking buffer. The label can be chosen from a variety of enzymes, including horseradish peroxidase (HRP), beta-galactosidase, alkaline phosphatase (ALP), and glucose oxidase. Sufficient time is allowed for specific binding to occur again, then the well is washed again to remove unbound conjugate, and a suitable substrate for the enzyme is added. Color is allowed to develop and the optical density of the contents of the well is determined visually or instrumentally (measured at an appropriate wave length). Conditions for performing ELISA assays are well-known in the art.

In another embodiment of an ELISA, a peptide or a collection or set of peptides disclosed herein is immobilized on a surface, such as a ninety-six-well ELISA plate A sample is then added and the assay proceeds as above.

In still other embodiments, a peptide or collection or set of peptides disclosed herein are electro- or dot-blotted onto nitrocellulose paper. Subsequently, a sample, such as a biological fluid (e.g., serum or plasma) is incubated with the blotted antigen, and antibody in the biological fluid is allowed to bind to the antigen(s). The bound antibody can then be detected, e.g., by standard immunoenzymatic methods or by visualization using metallic nanoparticles or nanoshells coupled to secondary antibodies or other antibody binding agents or combinations thereof.

It should be understood by one of skill in the art that any number of conventional protein assay formats, particularly immunoassay formats, may be designed to utilize the isolated peptides, and collections and sets of peptides disclosed herein. Thus, the use of the peptides is thus not limited by the selection of the particular assay format, and is believed to encompass assay formats that are known to those of skill in the art. To date most serology has been performed using singleplex ELISA, complement fixation or neutralization assays. More recently, Luminex-based systems have been employed that can address up to 100 antigenic targets simultaneously (i.e., 100 individual pathogens, 100 individual antigenic targets for one pathogen, or some variation thereof). Additionally, arrays are established that comprise spotted recombinant proteins expressed in vitro in E. coli, S. cerevesiae, baculoviruses, or cell-free, coupled transcription-translation.

One goal of the present disclosure is to automate the process of antibody detection and make it inexpensive, quick and accurate as well as detect exposure per se rather than to rigorously characterize humoral responses to specific pathogens.

One assay that meets these requirements is a programmable peptide array.

The peptide array capacity can be exploited to print multiple arrays per glass slide (configurations of 1, 3, 8, or 12 arrays can be printed).

Thus, one embodiment is a peptide microarray comprising peptides which are reactive with, and specific for one or more filovirus pathogens in Table 1 or EBOV vaccine and/or a collection or set of peptides shifted at least one residue across at least one peptide chosen from the group consisting of the peptides which is reactive with, and specific for one or more filovirus pathogens in Table 1 or EBOV vaccine.

A further embodiment is a peptide microarray comprising peptides which are reactive with, and specific for EBOV infection and/or EBOV vaccine chosen from the group consisting of the peptides listed in Tables 2, 3, 4 and 6 and combinations thereof, and/or a collection or set of peptides shifted at least one residue across at least one peptide chosen from the group consisting of the peptides listed in Tables 2, 3, 4 and 6 and combinations thereof.

Methods for Serological Detection of Exposure to Pathogens and/or Vaccination to EBOV

The present disclosure also includes methods and systems for the detection of exposure to antigens of certain pathogens, i.e., antibodies to pathogens, in any sample utilizing the various peptides, isolated and non-isolated, and peptide microarrays disclosed herein.

Suitable methods typically include: receiving or obtaining (e.g., from a patient) a sample of bodily fluid or tissue likely to contain antibodies; contacting (e.g., incubating or reacting) a sample to be assayed with a disclosed peptide or peptides, under conditions effective for the formation of a specific peptide-antibody complex (e.g., for specific binding of the peptide to the antibody); and assaying the contacted (reacted) sample for the presence of an antibody-peptide reaction (e.g., determining the amount of an antibody-peptide complex). The presence of an elevated amount of the antibody-peptide complex indicates that the subject was exposed to and infected by pathogen and/or has been vaccinated against EBOV.

Conditions for reacting peptides and antibodies so that they react specifically are well-known to those of skill in the art. See, e.g., Current Protocols in Immunology (Coligan et al., editors, John Wiley & Sons, Inc).

Once the disclosed peptide or peptides and sample antibody are permitted to react in a suitable medium, an assay is performed to determine the presence or absence of an antibody-peptide reaction. Any of the assays discussed herein can be used.

The methods and systems of the present disclosure may be used to detect exposure to antigens in research and clinical settings.

One sample for use in the methods is a biological sample. A biological sample may be obtained from a tissue of a subject or bodily fluid from a subject including but not limited to nasopharyngeal aspirate, blood, cerebrospinal fluid, saliva, serum, plasma, urine, sputum, bronchial lavage, pericardial fluid, or peritoneal fluid, or a solid such as feces. The subject may be any animal, particularly a vertebrate and more particularly a mammal, including, without limitation, a cow, dog, human, monkey, mouse, pig, or rat. In one embodiment, the subject is a human.

A sample may also be a research, clinical, or environmental sample, such as cells, cell culture, cell culture medium, and compositions for use as, or the development of pharmaceutical and therapeutic agents.

Additional applications include, without limitation, detection of the screening of blood products (e.g., screening blood products for infectious agents), biodefense, food safety, environmental contamination, forensics, and genetic-comparability studies. The present disclosure also provides methods and systems for detecting viral antibodies in cells, cell culture, cell culture medium and other compositions used for the development of pharmaceutical and therapeutic and immunomodulatory agents.

The present disclosure provides a method for detecting the exposure to antigens of one or more pathogens and/or infection by one or more filovirus pathogens in Table 1 and/or immunization by EBOV vaccine, in any sample, including the steps of: contacting the sample with any disclosed peptide or peptides which are reactive with, and specific for antibodies of one or more filovirus pathogens in Table 1 or EBOV vaccine peptides, under conditions sufficient for any antibodies in the sample and the peptides to bind; and visualizing and/or quantifying any bound antibody or antibodies to the peptides. The method may optionally include a step for washing any unbound sample.

The present disclosure provides a method for differentially detecting the exposure to antigens of one or more pathogens and/or infection by one or more filovirus pathogens in Table 1 and/or immunization by EBOV vaccine in any sample, including the steps of: contacting the sample with any of any disclosed peptide or peptides which are reactive with, and specific for antibodies of one or more filovirus pathogens in Table 1 or EBOV vaccine peptides, under conditions sufficient for any antibodies in the sample and the peptides to bind; and visualizing and/or quantifying any bound antibody or antibodies to the peptides. The method may optionally include a step for washing any unbound sample.

The present disclosure further provides a method for detecting the exposure to antigens of one or more pathogens and/or infection by one or more filovirus pathogens in Table 1 and/or immunization by EBOV vaccine, in any sample, including the steps of: contacting the sample with a peptide microarray as set forth in Table 1, under conditions sufficient for any antibodies in the sample and the peptides to bind; and visualizing and/or quantifying any bound antibody or antibodies to the peptides. The method may optionally include a step for washing any unbound sample.

The present disclosure further provides a method for differentially detecting the exposure to antigens of one or more pathogens and/or infection by one or more filovirus pathogens in Table 1 and/or immunization by EBOV vaccine, in any sample, including the steps of: contacting the sample with a peptide microarray as set forth in Table 1, under conditions sufficient for any antibodies in the sample and the peptides to bind; and visualizing and/or quantifying any bound antibody or antibodies to the peptides. The method may optionally include a step for washing any unbound sample.

The present disclosure also provides for a method of identifying a subject who has been infected with EBOV and/or survived EBOV, in any sample, including the steps of: contacting the sample with at least one peptide chosen from the group consisting of the peptides listed in Table 2 and 3, and combinations and/or a collection or set of peptides shifted at least one residue across at least one peptide chosen from the group consisting of the peptides listed in Tables 2 and 3, and combinations thereof under conditions sufficient for any antibodies in the sample and the peptides to bind; and visualizing and/or quantifying any bound antibody or antibodies to the peptides. The method may optionally include a step for washing any unbound sample.

The present disclosure also provides for a method of identifying a subject who has been vaccinated against EBOV, in any sample, including the steps of: contacting the sample with at least one peptide chosen from the group consisting of the peptides listed in Tables 2, 4 and 6, and combinations thereof and/or a collection or set of peptides shifted at least one residue across at least one peptide chosen from the group consisting of the peptides listed in Tables 2, 4 and 6, and combinations thereof under conditions sufficient for any antibodies in the sample and the peptides to bind; and visualizing and/or quantifying any bound antibody or antibodies to the peptides. The method may optionally include a step for washing any unbound sample.

A further embodiment is a method of identifying subject who have had a natural infection to EBOV and have been vaccinated again EBOV, in any sample, including the steps of: contacting the sample with at least one peptide chosen from the group consisting of the peptides listed in Tables 2, 3, 4 and 6, and combinations thereof and/or a collection or set of peptides shifted at least one residue across at least one peptide chosen from the group consisting of the peptides listed in Tables 2, 3, 4 and 6, and combinations thereof under conditions sufficient for any antibodies in the sample and the peptides to bind; and visualizing and/or quantifying any bound antibody or antibodies to the peptides. The method may optionally include a step for washing any unbound sample.

The present disclosure also provides for a method of identifying a subject who has been infected with EBOV and/or survived EBOV, in any sample, including the steps of: contacting the sample with a peptide microarray comprising at least one peptide chosen from the group consisting of the peptides listed in Table 2 and 3, and combinations thereof, and/or a collection or set of peptides shifted at least one residue across at least one peptide chosen from the group consisting of the peptides listed in Tables 2 and 3, and combinations thereof under conditions sufficient for any antibodies in the sample and the peptides to bind; and visualizing and/or quantifying any bound antibody or antibodies to the peptides. The method may optionally include a step for washing any unbound sample.

The present disclosure also provides for a method of identifying a subject who has been vaccinated against EBOV, in any sample, including the steps of: contacting the sample with a peptide microarray comprising at least one peptide chosen from the group consisting of the peptides listed in Tables 2, 4 and 6, and combinations thereof and/or a collection or set of peptides shifted at least one residue across at least one peptide chosen from the group consisting of the peptides listed in Tables 2, 4 and 6, and combinations thereof under conditions sufficient for any antibodies in the sample and the peptides to bind; and visualizing and/or quantifying any bound antibody or antibodies to the peptides. The method may optionally include a step for washing any unbound sample.

A further embodiment is a method of identifying subject who have had a natural infection to EBOV and have been vaccinated again EBOV, in any sample, including the steps of: contacting the sample with a peptide microarray comprising at least one peptide chosen from the group consisting of the peptides listed in Tables 2, 3, 4 and 6, and combinations thereof and/or a collection or set of peptides shifted at least one residue across at least one peptide chosen from the group consisting of the peptides listed in Tables 2, 3, 4 and 6, and combinations thereof under conditions sufficient for any antibodies in the sample and the peptides to bind; and visualizing and/or quantifying any bound antibody or antibodies to the peptides. The method may optionally include a step for washing any unbound sample.

In some embodiments, the peptides listed in Table 6 are VSV10 and VSV11.

Any method of detection discussed herein or known in the art can be used for visualizing and/or quantifying the bound antibodies.

### Kits

The present disclosure also includes reagents and kits for practicing the disclosed methods. These reagents and kits may vary.

### EXAMPLES

The present invention may be better understood by reference to the following non-limiting examples, which are presented in order to more fully illustrate the preferred embodiments of the invention. They should in no way be construed to limit the broad scope of the invention.

### Example 1—West African Peptide Array

The platform described herein for epitope discovery is a programmable peptide microarray that can accommodate up to 6 million distinct linear peptides on a 75 mm×26 mm slide. The array can also be divided into 12 subarrays, each containing approximately 380,000 12-mer peptides. The 12-mer format is based on the observation that serum antibodies bind linear peptide sequences ranging from 5 to 9 amino acid (aa) and bind most efficiently when targets are flanked by additional aa [1]. To enable differential detection of antibodies specific for different filoviruses infections and VSV-EBOV-GP vaccinations, a custom NCBI GenBank and VIPR database for full proteomes of filoviruses and lyssaviruses, proteomes from reference genomes of arboviruses, selective immunogenic proteins of Mycobacterium tuberculosis, Salmonella typhi, Plasmodium falciparum, and proteomes of reference genomes of mumps, mesles and rubella for positive controls was created (Table 1). For each virus selected, all available protein sequences available before January, 2020 were downloaded from the NCBI and VIPR protein databases. Then a database was created comprising overlapping 12-mer peptides that tiled the whole proteome of each of these agents with 11 amino acid (aa) overlap in a sliding window pattern. The selected peptide sequences were passed through a redundancy filter to yield 390894 unique peptide sequences. Redundant peptides were excluded prior to synthesis. The individual peptides in the library were printed in random positions on the peptide array to minimize the impact of locational bias (Table 1).

TABLE 1
Peptide Array Features

| | | | | Number of | | |
|---|---|---|---|---|---|---|
| | | | | protein | Total 12 - aa | Unique 12- |
| | Name | Type | Entry source | sequences | peptides | aa peptides |
| 1 | Filoviridae | Virus | NCBI proteome database | 22586 | 13560792 | 114567 |
| 2 | Zaire EBOV Mayinga | Virus | Reference sequences | 9 | 5394 | 4826 |
| | (VACCINE) | | | | | |
| 3 | Vesicular stomatitis | Virus | Reference sequences | 5 | 3481 | 3481 |
| | Indiana virus (vector) | | | | | |
| 4 | Lyssavirus | Virus | NCBI proteome database | 35954 | 14443831 | 198813 |
| 5 | Dengue viruses (1-4) | Virus | Reference sequences | 4 | 13516 | 12183 |
| 6 | Yellow fever virus | Virus | Reference sequences | 1 | 3400 | 3400 |
| 7 | West Nile virus | Virus | Reference sequences | 2 | 4599 | 3462 |
| 8 | Semliki Forest virus | Virus | Reference sequences | 3 | 4481 | 3688 |
| 9 | Rift valley fever virus | Virus | Reference sequences | 4 | 3755 | 3755 |
| 10 | Chikungunya virus | Virus | Reference sequences | 2 | 3700 | 3700 |
| 11 | O'nyong'nyong virus | Virus | Reference sequences | 3 | 4551 | 3766 |
| 12 | Zika virus | Virus | Reference sequences | 1 | 3408 | 3408 |
| 13 | Rubella virus | Virus | Reference sequences | 2 | 3157 | 3157 |
| 14 | Mumps virus | Virus | Reference sequences | 62 | 35535 | 6895 |
| 15 | Measles morbillivirus | Virus | Reference sequences | 37 | 24828 | 5698 |
| 16 | Mycobacterium | Bacteria | Selective immunogenic proteins | 13 | 4012 | 4012 |
| | tuberculosis | | | | | |
| 17 | Salmonella Typhi | Bacteria | Selective immunogenic proteins | 7 | 3196 | 3196 |
| 18 | Plasmodium falciparum | protozoan | Selective immunogenic proteins | 14 | 7216 | 7162 |
| 19 | SARS - CoV - 2 | Virus | Selective peptides | 163 | 804 | 804 |
| 20 | coronaviruses | Virus | Selective peptides | 53 | 428 | 375 |
| | Other Human | | | | | |
| 21 | Human enteroviruses | Virus | Selective peptides | 6 | 46 | 46 |
| 22 | non - specific peptides | NA | | | | 500 |
| | Random scrammbled | | | | | |

| | |
|---|---|
| Total 12- aa unique peptides in each sub-array | 390894 |

### Example 2—Identification of EBOV Peptides Useful for Detection and Diagnosis

### Experimental Design and Samples:

### Non-Human Primates

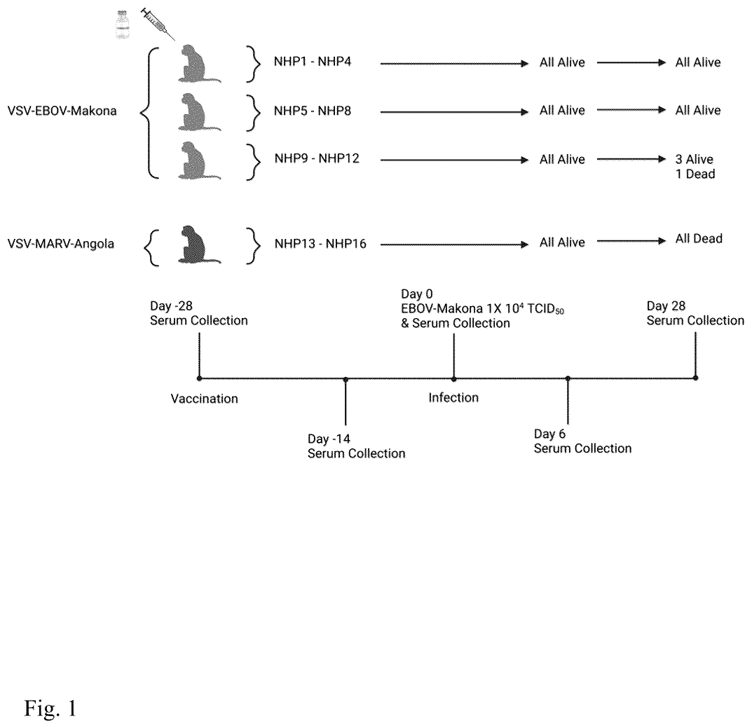

72 plasma samples were collected from 16 monkeys that were vaccinated with VSV-EBOV-GP vaccine or VSV-Marburg-GP vaccine and challenged with EBOV infection (Makona strain). Monkeys were vaccinated with different dosage (10⁰, 10¹, and 10⁶ PFU of VSV-EBOV-GP) and control monkeys were with PFU 10⁷ of VSV-MARV-GP. Blood samples were collected at different time points; at the time of vaccination (D-28), 14 days post vaccination (D-14), at the time of challenge after 28 days of vaccination (D0), 6 days post infection (D6), 14 days post infection (D14), 28 days post infection (D28). All the NHPs were challenged with EBOV-Makona at 1×10⁴ TCID (FIG. 1).

### Immunoreactive EBOV Epitopes

Immunogenic epitopes were mapped against EBOV proteome database (2).

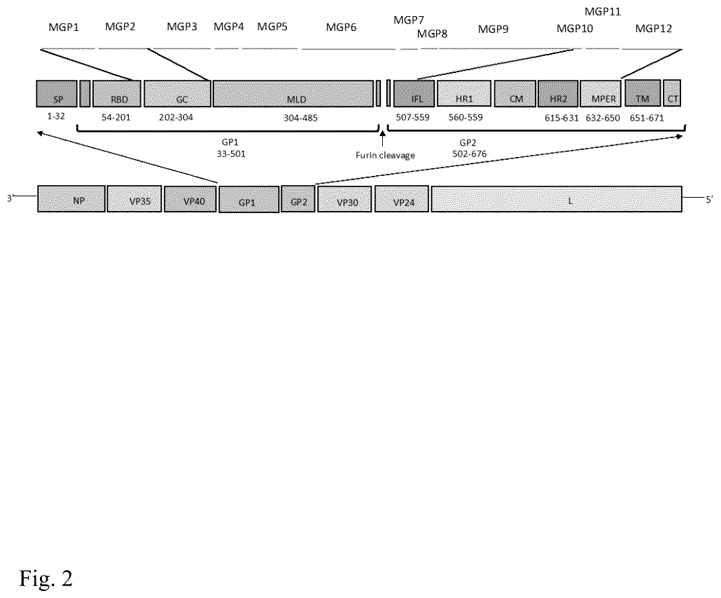

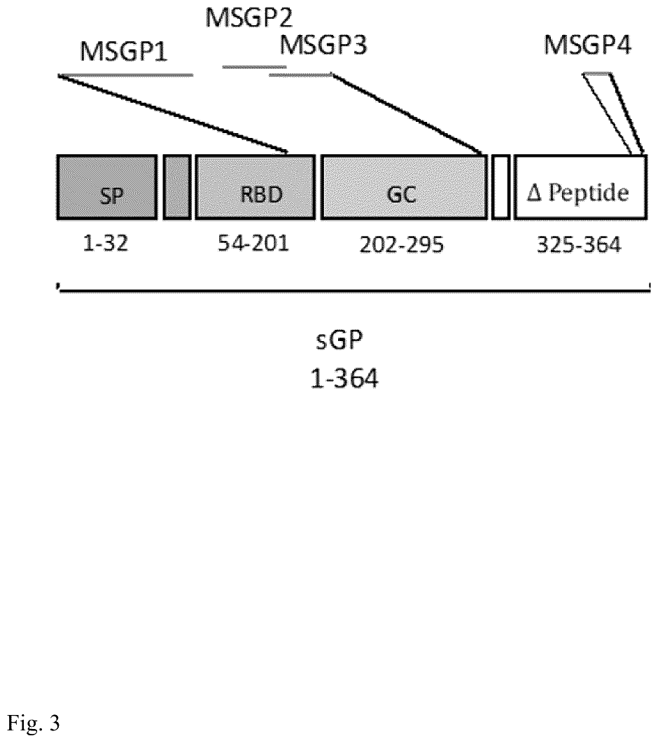

12 highly reactive epitopes were identified in GP (FIG. 2), and 4 in sGP (FIG. 3) that include 7 epitopes in the mucin like domain (MLD) region.

Two immunoreactive epitopes, MGP1 and MGP2, were identified in the glycan cap (GC) region near receptor binding domain (RBD), that is common to soluble glycoprotein (sGP) and small soluble glycoprotein (ssGP). In previous studies MLD has not been reported as a protective neutralizing epitope binding region. An immunoreactive epitope, MGP12, was identified in the region of membrane proximal external region (MPER). Immunoreactivity to MPER has been associated with protection in EBOV-infection survivors and is postulated to include neutralizing antibody epitope sites. The immunoreactive GP and sGP epitopes are listed according to their locations from N terminal to C Terminal in Table 2.

TABLE 2
Immunoreactive epitopes in the EBOV-GP and sGP detected using plasma from monkeys immunized with VSV-EBOV-Makona-GP and subsequently infected
with EBOV-Makona (SEQ ID NOS: 2-17, from top to bottom).

| | Epi- | | | | | | | | | | | | | | | | | | |
|---|---|---|---|---|---|---|---|---|---|---|---|---|---|---|---|---|---|---|---|
| | tope | | 10\hat{ }7(VSV-MARV-GP) | | | | 1 (VSV-EBOV-GP) | | | 10(VSV-EBOV-GP) | | | | | 10\hat{ }6(VSV-EBOV-GP) | | | | |
| Epi- | Se- | Pro- | NHP13-NHP16 | | | | NHP9-NHP12 | | | NHP5-NHP8 | | | | | NHP1-NHP4 | | | | |
| tope | quence | tein | D-28 | D-14 | D0 | D6 | D-28 | D6 | D28 | D-28 | D-14 | D0 | D6 | D28 | D-28 | D-14 | D0 | D6 | D28 |
| MGP1 | LILPQ | GP1, 2 | 0/4 | 0/4 | 0/4 | 0/4 | 0/4 | 3/4 | 3/3 | 0/4 | 0/4 | 0/4 | 2/4 | 3/4 | 0/4 | 2/4 | 0/4 | 4/4 | 4/4 |
| | AKKDF | | (0%) | (0%) | (0%) | (0%) | (0%) | (75%) | (100%) | (0%) | (0%) | (0%) | (50%) | (75%) | (0%) | (50%) | (0%) | (100%) | (100%) |
| | FSSHP | | | | | | | | | | | | | | | | | | |
| | LREPV | | | | | | | | | | | | | | | | | | |
| | NATED | | | | | | | | | | | | | | | | | | |
| | P | | | | | | | | | | | | | | | | | | |
| MGP2 | TTGKL | GP1, 2 | 0/4 | 0/4 | 0/4 | 0/4 | 0/4 | 2/4 | 2/3 | 0/4 | 0/4 | 0/4 | 2/4 | 3/4 | 0/4 | 0/4 | 0/4 | 4/4 | 4/4 |
| | IWKVN | | (0%) | (0%) | (0%) | (0%) | (0%) | (50%) | (66%) | (0%) | (0%) | (0%) | (50%) | (75%) | (0%) | (0%) | (0%) | (100%) | (100%) |
| | PEIDT | | | | | | | | | | | | | | | | | | |
| | TIGEW | | | | | | | | | | | | | | | | | | |
| MGP3 | EIDTT | IGP1, 2 | 0/4 | 0/4 | 0/4 | 0/4 | 0/4 | 4/4 | 3/3 | 0/4 | 1/4 | 0/4 | 4/4 | 4/4 | 0/4 | 2/4 | %)2/4 | 4/4 | 4/4 |
| | IGEWA | | (0%) | (0%) | (0%) | (0%) | (0%) | (100%) | (100%) | (0%) | (25%) | (0%) | (100%) | (100%) | (0%) | (50%) | (50 | (100%) | (100%) |
| | FWETK | | | | | | | | | | | | | | | | | | |
| | KNLTR | | | | | | | | | | | | | | | | | | |
| | KIR | | | | | | | | | | | | | | | | | | |
| MGP4 | AVSNG | GP1, 2 | 0/4 | 0/4 | 0/4 | 0/4 | 0/4 | 4/4 | 3/3 | 0/4 | 0/4 | 0/4 | 4/4 | 4/4 | 0/4 | 1/4 | 0/4 | 4/4 | 4/4 |
| | PKNIS | | (0%) | (0%) | (0%) | (0%) | (0%) | (100%) | (100%) | (0%) | (0%) | (0%) | (100%) | (100%) | (0%) | (25%) | (0%) | (100%) | (100%) |
| | GQSPA | | | | | | | | | | | | | | | | | | |
| | RTSSD | | | | | | | | | | | | | | | | | | |
| | PETNT | | | | | | | | | | | | | | | | | | |
| | TNE | | | | | | | | | | | | | | | | | | |
| MGP5 | NTTNE | GP1, 2 | 0/4 | 0/4 | 0/4 | 0/4 | 0/4 | 4/4 | 3/3 | 0/4 | 0/4 | 0/4 | 3/4 | 3/4 | 0/4 | 0/4 | 0/4 | 4/4 | 4/4 |
| | DHKIM | | (0%) | (0%) | (0%) | (0%) | (0%) | (100%) | (100%) | (0%) | (0%) | (0%) | (75%) | (75%) | (0%) | (0%) | (0%) | (100%) | (100%) |
| | ASENS | | | | | | | | | | | | | | | | | | |
| | SAMV | | | | | | | | | | | | | | | | | | |
| MGP6 | ASENS | GP1, 2 | 0/4 | 0/4 | 0/4 | 0/4 | 0/4 | 4/4 | 3/3 | 0/4 | 0/4 | 0/4 | 4/4 | 4/4 | 0/4 | 2/4 | 2/4 | 4/4 | 4/4 |
| | SAMVQ | | (0%) | (0%) | (0%) | (0%) | (0%) | (100%) | (100%) | (0%) | (0%) | (0%) | (100%) | (100%) | (0%) | (50%) | (50%) | (100%) | (100%) |
| | VHSQG | | | | | | | | | | | | | | | | | | |
| | RKAAV | | | | | | | | | | | | | | | | | | |
| | SHLTT | | | | | | | | | | | | | | | | | | |
| | LAT | | | | | | | | | | | | | | | | | | |
| MGP7 | DNDST | GP1, 2 | 0/4 | 0/4 | 0/4 | 0/4 | 0/4 | 4/4 | 3/3 | 0/4 | 0/4 | 0/4 | 4/4 | 4/4 | 0/4 | 0/4 | 1/4 | 4/4 | 4/4 |
| | ASDTP | | (0%) | (0%) | (0%) | (0%) | (0%) | (100%) | (100%) | (0%) | (0%) | (0%) | (100%) | (100%) | (0%) | (0%) | (25%) | (100%) | (100%) |
| | PADNS | | | | | | | | | | | | | | | | | | |
| | THNTP | | | | | | | | | | | | | | | | | | |
| | VYKLD | | | | | | | | | | | | | | | | | | |
| | ISEAT | | | | | | | | | | | | | | | | | | |
| | QVGQH | | | | | | | | | | | | | | | | | | |
| | HRRA | | | | | | | | | | | | | | | | | | |
| MGP8 | NTSKS | GP1, 2 | 0/4 | 2/4 | 1/4 | 0/4 | 2/4 | 4/4 | 3/3 | 1/4 | 0/4 | 0/4 | 4/4 | 4/4 | 2/4 | 1/4 | 2/4 | 4/4 | 4/4 |
| | ADSLD | | (0%) | (50%) | (25%) | (0%) | (50%) | (100%) | (100%) | (25%) | (0%) | (0%) | (100%) | (100%) | (50%) | (25%) | (50%) | (100%) | (100%) |
| | LPATT | | | | | | | | | | | | | | | | | | |
| | AAGPL | | | | | | | | | | | | | | | | | | |
| | KAENT | | | | | | | | | | | | | | | | | | |
| MGP9 | ETAGN | GP1, 2 | 0/4 | 1/4 | 0/4 | 1/4 | 0/4 | 4/4 | 3/3 | 1/4 | 0/4 | 0/4 | 4/4 | 4/4 | 0/4 | 2/4 | 2/4 | 4/4 | 4/4 |
| | NNTHH | | (0%) | (25%) | (0%) | (25%) | (0%) | (100%) | (100%) | (25%) | (0%) | (0%) | (100%) | (100%) | (0%) | (50%) | (50%) | (100%) | (100%) |
| | QDTGE | | | | | | | | | | | | | | | | | | |
| | ESASS | | | | | | | | | | | | | | | | | | |
| | GKLGL | | | | | | | | | | | | | | | | | | |
| | ITNTI | | | | | | | | | | | | | | | | | | |
| | AGVAG | | | | | | | | | | | | | | | | | | |
| | LITGG | | | | | | | | | | | | | | | | | | |
| | RRTRR | | | | | | | | | | | | | | | | | | |
| | EVI | | | | | | | | | | | | | | | | | | |
| MGP10 | GLAWI | GP1, 2 | 0/4 | 0/4 | 0/4 | 0/4 | 0/4 | 2/4 | 3/3 | 0/4 | 0/4 | 0/4 | 0/4 | 0/4 | 0/4 | 0/4 | 0/4 | 2/4 | 4/4 |
| | PYFGP | | (0%) | (0%) | (0%) | (0%) | (0%) | (50%) | (100%) | (0%) | (0%) | (0%) | (0%) | (0%) | (0%) | (0%) | (0%) | (50%) | (100%) |
| | AAEGI | | | | | | | | | | | | | | | | | | |
| | Y | | | | | | | | | | | | | | | | | | |
| MGP11 | RTFSI | GP1, 2 | 0/4 | 0/4 | 0/4 | 0/4 | 0/4 | 1/4 | 3/3 | 0/4 | 0/4 | 0/4 | 0/4 | 2/4 | 0/4 | 0/4 | 0/4 | 0/4 | 4/4 |
| | LNRKA | | (0%) | (0%) | (0%) | (0%) | (0%) | (25%) | (100%) | (0%) | (0%) | (0%) | (0%) | (50%) | (0%) | (0%) | (0%) | (0%) | (100%) |
| | IDFLL | | | | | | | | | | | | | | | | | | |
| | QRWGG | | | | | | | | | | | | | | | | | | |
| | TCH | | | | | | | | | | | | | | | | | | |
| MGP12 | DKIDQ | GP1, 2 | 0/4 | 0/4 | 0/4 | 0/4 | 0/4 | 4/4 | 3/3 | 0/4 | 0/4 | 0/4 | 3/4 | 4/4 | 0/4 | 0/4 | 0/4 | 4/4 | 4/4 |
| | IIHDF | | (0%) | (0%) | (0%) | (0%) | (0%) | (100%) | (100%) | (0%) | (0%) | (0%) | (75%) | (100%) | (0%) | (0%) | (0%) | (100%) | (100%) |
| | VDKTL | | | | | | | | | | | | | | | | | | |
| | PDQGD | | | | | | | | | | | | | | | | | | |
| | NDNWW | | | | | | | | | | | | | | | | | | |
| | TGWRQ | | | | | | | | | | | | | | | | | | |
| | W | | | | | | | | | | | | | | | | | | |
| MSGP1 | FLILP | sGP | 0/4 | 0/4 | 0/4 | 0/4 | 0/4 | 3/4 | 3/3 | 0/4 | 0/4 | 0/4 | 2/4 | 3/4 | 0/4 | 2/4 | 0/4 | 4/4 | 4/4 |
| | QAKKD | | (0%) | (0%) | (0%) | (0%) | (0%) | (75%) | (100%) | (0%) | (0%) | (0%) | (50%) | (75%) | (0%) | (50%) | (0%) | (100%) | (100%) |
| | FFSSH | | | | | | | | | | | | | | | | | | |
| | PLREP | | | | | | | | | | | | | | | | | | |
| | VNATE | | | | | | | | | | | | | | | | | | |
| | DP | | | | | | | | | | | | | | | | | | |
| MSGP2 | TTGKL | sGP | 0/4 | 0/4 | 0/4 | 0/4 | 0/4 | 2/4 | 2/3 | 0/4 | 0/4 | 0/4 | 2/4 | 3/4 | 0/4 | 0/4 | 0/4 | 4/4 | 4/4 |
| | IWKVN | | (0%) | (0%) | (0%) | (0%) | (0%) | (50%) | (66%) | (0%) | (0%) | (0%) | (50%) | (75%) | (0%) | (0%) | (0%) | (100%) | (100%) |
| | PEIDT | | | | | | | | | | | | | | | | | | |
| | TIGEW | | | | | | | | | | | | | | | | | | |
| MSGP3 | EIDTT | sGP | 0/4 | 0/4 | 0/4 | 0/4 | 0/4 | 4/4 | 3/3 | 0/4 | 2/4 | 0/4 | 4/4 | 4/4 | 0/4 | 2/4 | 1/4 | 4/4 | 4/4 |
| | IGEWA | | (0%) | (0%) | (0%) | (0%) | (0%) | (0%) | (100%) | (0%) | (50%) | (0%) | (100%) | (100%) | (0%) | (50%) | (25%) | (100%) | (100%) |
| | FWETK | | | | | | | | | | | | | | | | | | |
| | KTSLE | | | | | | | | | | | | | | | | | | |
| MSGP4 | FKCTV | SGP | 0/4 | 0/4 | 0/4 | 0/4 | 0/4 | 0/4 | 2/3 | 0/4 | 0/4 | 0/4 | 0/4 | 1/4 | 0/4 | 0/4 | 0/4 | 0/4 | 4/4 |
| | KEGKL | | (0%) | (0%) | (0%) | (0%) | (0%) | (0%) | (66%) | (0%) | (0%) | (0%) | (0%) | (25%) | (0%) | (0%) | (0%) | (0%) | (100%) |
| | QCRI | | | | | | | | | | | | | | | | | | |

Immunoreactive epitopes were also identified in the nucleoprotein, VP35, VP40, VP24, and polymerase proteins (Table 3). Most of the epitopes started showing reactivity after 6 days post-challenge and reached maximum reactivity at 28 days post-challenge.

TABLE 3
Immunoreactive epitopes in the EBOV nucleoprotein (NP), viral protein 35 (VP35), viral protein 40 (VP40), viral protein 24 (VP24) and
polymerase (L) detected using plasma from monkeys immunized with VSV-EBOV-Makona-GP and subsequently infected with EBOV-Makona.
(SEQ ID NOS: 18-36, from top to bottom).

| | Epi- | | | | | | | | | | | | | | | | | | |
|---|---|---|---|---|---|---|---|---|---|---|---|---|---|---|---|---|---|---|---|
| | tope | | 10\hat{ }7(VSV-MARV-GP) | | | | 1 (VSV-EBOV-GP) | | | | 10(VSV-EBOV-GP) | | | | 10\hat{ }6(VSV-EBOV-GP) | | | | |
| Epi- | Se- | Pro- | NHP13-NHP16 | | | | NHP9-NHP12 | | | | NHP5-NHP8 | | | | NHP1-NHP4 | | | | |
| tope | quence | tein | D-28 | D-14 | D0 | D6 | D-28 | D6 | D28 | D-28 | D-14 | D0 | D6 | D28 | D-28 | D-14 | D0 | D6 | D28 |
| MNP1 | ASLPKTS | NP | 1/4 | 0/4 | 0/4 | 1/4 | 0/4 | 0/4 | 2/4 | 0/4 | 0/4 | 0/4 | 2/4 | 2/4 | 0/4 | 0/4 | 0/4 | 0/4 | 4/4 |
| | GHYDDDD | | (25%) | (0%) | (0%) | (25%) | (0%) | (0%) | (50%) | (0%) | (0%) | (0%) | (50%) | (50%) | (0%) | (0%) | (0%) | (0%) | (100%) |
| | DIPFPGP | | | | | | | | | | | | | | | | | | |
| | INDDDNP | | | | | | | | | | | | | | | | | | |
| | GHQD | | | | | | | | | | | | | | | | | | |
| MNP2 | TIPDVVV | NP | 0/4 | 0/4 | 0/4 | 0/4 | 0/4 | 0/4 | 3/4 | 0/4 | 0/4 | 0/4 | 0/4 | 1/4 | 0/4 | 0/4 | 0/4 | 0/4 | 4/4 |
| | DPDDGGY | | (0%) | (0%) | (0%) | (0%) | (0%) | (0%) | (75%) | (0%) | (0%) | (0%) | (0%) | (25%) | (0%) | (0%) | (0%) | (0%) | (100%) |
| | GEYQSYS | | | | | | | | | | | | | | | | | | |
| MNP3 | SENGMSA | NP | 0/4 | 0/4 | 0/4 | 0/4 | 0/4 | 0/4 | 2/4 | 0/4 | 0/4 | 0/4 | 0/4 | 1/4 | 0/4 | 0/4 | 0/4 | 0/4 | 4/4 |
| | PDDLVLF | | (0%) | (0%) | (0%) | (0%) | (0%) | (0%) | (50%) | (0%) | (0%) | (0%) | (0%) | (25 | (0%) | (0%) | (0%) | (0%) | (100%) |
| | DLDEDDE | | | | | | | | | | | | | | | | | | |
| | DTKPVPN | | | | | | | | | | | | | | | | | | |
| | RS | | | | | | | | | | | | | | | | | | |
| MNP4 | GQQKNSQ | NP | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 3/4 | 3/4 |
| | KGQHTEG | | (0%) | (0%) | (0%) | (0%) | (0%) | (0%) | (0%) | (0%) | (0%) | (0%) | (0%) | (0%) | (0%) | (0%) | (0%) | (75%) | (75%) |
| | RQTQ | | | | | | | | | | | | | | | | | | |
| MNP5 | TDNDRRN | NP | 1/4 | 0/4 | 0/4 | 2/4 | 0/4 | 0/4 | 0/4 | 0/4 | 0/4 | 0/4 | 0/4 | 1/4 | 0/4 | 0/4 | 0/4 | 2/4 | 3/4 |
| | EPSGSTS | | (25%) | (0%) | (0%) | (50%) | (0%) | (0%) | (0%) | (0%) | (0%) | (0%) | (0%) | (25%) | (0%) | (0%) | (0%) | (50%) | (75%) |
| | PRML | | | | | | | | | | | | | | | | | | |
| MNP6 | PTVAPPA | INP | 0/4 | 0/4 | 0/4 | 0/4 | 0/4 | 1/4 | 2/4 | 1/4 | 0/4 | 0/4 | 1/4 | 2/4 | 0/4 | 0/4 | 0/4 | 0/4 | 3/4 |
| | PVYRDHS | | (0%) | (0%) | (0%) | (0%) | (0%) | (25%) | (50%) | (25%) | (0%) | (0%) | (25%) | (50%) | (0%) | (0%) | (0%) | (0%) | (75%) |
| | EKKE | | | | | | | | | | | | | | | | | | |
| MNP7 | EQQDQDH | NP | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 3/4 |
| | IQEARNQ | | (0%) | (0%) | (0%) | (0%) | (0%) | (0%) | (0%) | (0%) | (0%) | (0%) | (0%) | (0%) | (0%) | (0%) | (0%) | (0%) | (75%) |
| | D | | | | | | | | | | | | | | | | | | |
| MVP35E1 | MPGPELS | VP35 | 0/4 | 0/4 | 0/4 | 0/4 | 0/4 | 0/4 | 2/4 | 0/4 | 0/4 | 0/4 | 0/4 | 1/4 | 0/4 | 0/4 | 0/4 | 0/4 | 4/4 |
| | GWISEQL | | (0%) | (0%) | (0%) | (0%) | (0%) | (0%) | (50%) | (0%) | (0%) | (0%) | (0%) | (25%) | (0%) | (0%) | (0%) | (0%) | (100%) |
| | MTG | | | | | | | | | | | | | | | | | | |
| MVP35E2 | DIFCDIE | VP35 | 0/4 | 0/4 | 0/4 | 0/4 | 0/4 | 0/4 | 1/4 | 0/4 | 0/4 | 0/4 | 0/4 | 2/4 | 0/4 | 0/4 | 0/4 | 0/4 | 4/4 |
| | NNPGLCY | | (0%) | (0%) | (0%) | (0%) | (0%) | (0%) | (25%) | (0%) | (0%) | (0%) | (0%) | (50%) | (0%) | (0%) | (0%) | (0%) | (100%) |
| | ASQ | | | | | | | | | | | | | | | | | | |
| MVP35E3 | ATAAATE | VP35 | 0/4 | 0/4 | 0/4 | 0/4 | 0/4 | 0/4 | 2/4 | 0/4 | 0/4 | 0/4 | 0/4 | 2/4 | 0/4 | 0/4 | 0/4 | 0/4 | 4/4 |
| | AYWAEHG | | (0%) | (0%) | (0%) | (0%) | (0%) | (0%) | (50%) | (0%) | (0%) | (0%) | (0%) | (50%) | (0%) | (0%) | (0%) | (0%) | (100%) |
| | QPPPGPS | | | | | | | | | | | | | | | | | | |
| MVP35E4 | IRGKIEE | VP35 | 0/4 | 0/4 | 0/4 | 0/4 | 0/4 | 0/4 | 2/4 | 0/4 | 0/4 | 0/4 | 0/4 | 2/4 | 0/4 | 0/4 | 0/4 | 1/4 | 4/4 |
| | HGQPPPG | | (0%) | (0%) | (0%) | (0%) | (0%) | (0%) | (50%) | (0%) | (0%) | (0%) | (0%) | (50%) | (0%) | (0%) | (0%) | (25%) | (100%) |
| | PSLYEES | | | | | | | | | | | | | | | | | | |
| | A | | | | | | | | | | | | | | | | | | |
| MVP35E5 | ETVPQSV | VP35 | 0/4 | 0/4 | 0/4 | 0/4 | 0/4 | 0/4 | 3/4 | 0/4 | 0/4 | 0/4 | 1/4 | %)2/4 | 0/4 | 0/4 | 0/4 | 0/4 | 4/4 |
| | REAFNNL | | (0%) | (0%) | (0%) | (0%) | (0%) | (0%) | (75%) | (0%) | (0%) | (0%) | (25%) | (50 | (0%) | (0%) | (0%) | (0%) | (100%) |
| | DSTTSLT | | | | | | | | | | | | | | | | | | |
| | E | | | | | | | | | | | | | | | | | | |
| MVP35E6 | DSTTSLT | VP35 | 0/4 | 0/4 | 0/4 | 0/4 | 0/4 | 0/4 | 2/4 | 0/4 | 0/4 | 0/4 | 1/4 | %)2/4 | 0/4 | 0/4 | 0/4 | 1/4 | 4/4 |
| | EENFGKP | | (0%) | (0%) | (0%) | (0%) | (0%) | (0%) | (50%) | (0%) | (0%) | (0%) | (25%) | (50 | (0%) | (0%) | (0%) | (25%) | (100%) |
| | DISAKDL | | | | | | | | | | | | | | | | | | |
| | RNIMY | | | | | | | | | | | | | | | | | | |
| MV40E1 | YMEAIYP | VP40 | 0/4 | 0/4 | 0/4 | 0/4 | 0/4 | 1/4 | 2/4 | 0/4 | 0/4 | 0/4 | 1/4 | %)0/4 | 0/4 | 0/4 | 0/4 | 1/4 | 4/4 |
| | ARSNSTI | | (0%) | (0%) | (0%) | (0%) | (0%) | (25%) | (50%) | (0%) | (0%) | (0%) | (25%) | (0 | (0%) | (0%) | (0%) | (25%) | (100%) |
| | AR | | | | | | | | | | | | | | | | | | |
| MV40E2 | LPKYIGL | VP40 | 0/4 | 0/4 | 0/4 | 0/4 | 0/4 | 0/4 | 1/4 | 0/4 | 0/4 | 0/4 | 0/4 | 1/4 | 0/4 | 0/4 | 0/4 | 0/4 | 3/4 |
| | DPVAPGD | | (0%) | (0%) | (0%) | (0%) | (0%) | (0%) | (25%) | (0%) | (0%) | (0%) | (0%) | (25%) | (0%) | (0%) | (0%) | (0%) | (75%) |
| | LTMVI | | | | | | | | | | | | | | | | | | |
| MV40E3 | VITQDCD | VVP40 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 3/4 |
| | TCHSPAS | | (0%) | (0%) | (0%) | (0%) | (0%) | (0%) | (0%) | (0%) | (0%) | (0%) | (0%) | (0%) | (0%) | (0%) | (0%) | (0%) | (75%) |
| | LP | | | | | | | | | | | | | | | | | | |
| MVP24E1 | FPHLFQN | VP24 | 1/4 | 0/4 | 0/4 | 1/4 | 0/4 | 2/4 | 1/4 | 1/4 | 0/4 | 0/4 | 2/4 | 3/4 | 0/4 | 0/4 | 0/4 | 3/4 | 4/4 |
| | PNSTIES | | (25%) | (0%) | (0%) | (25%) | (0%) | (50%) | (25%) | (25%) | (0%) | (0%) | (50%) | (75%) | (0%) | (0%) | (0%) | (75%) | (100%) |
| | PLWAL | | | | | | | | | | | | | | | | | | |
| ML1 | ELIYDNN | L | 0/4 | 0/4 | 0/4 | 0/4 | 2/4 | 2/4 | 0/4 | 1/4 | 0/4 | 0/4 | 2/4 | 1/4 | 0/4 | 0/4 | 0/4 | 3/4 | 3/4 |
| | PLKGGLN | | (0%) | (0%) | (0%) | (0%) | (50%) | (50%) | (0%) | (25%) | (0%) | (0%) | (50%) | (25%) | (0%) | (0%) | (0%) | (75%) | (75%) |
| | CNISFD | | | | | | | | | | | | | | | | | | |
| ML2 | TGIVSSM | L | 0/4 | 0/4 | 0/4 | 0/4 | 0/4 | 1/4 | 1/4 | 0/4 | 0/4 | 0/4 | 1/4 | 1/4 | 0/4 | 0/4 | 0/4 | 3/4 | 4/4 |
| | HYKLDEV | | (0%) | (0%) | (0%) | (0%) | (0%) | (25%) | (25%) | (0%) | (0%) | (0%) | (25%) | (25%) | (0%) | (0%) | (0%) | (75%) | (100%) |
| | LWEIEN | | | | | | | | | | | | | | | | | | |

### Immunoreactive VSV Epitopes

Using plasma from monkeys vaccinated with VSV-EBOV-Makona-GP, 9 immunoreactive epitopes were identified in the VSV vector: 4 epitopes in the phosphoprotein and nucleocapsid protein, 4 epitopes in the polymerase and 1 in matrix protein (Table 4).

TABLE 4
Immunoreactive epitopes in the VSV-EBOV-Makona-GP vector detected using plasma from monkeys immunized with VSV-EBOV-Makona-GP.
(SEQ ID NOS: 37-45, from top to bottom).

Epi-

| | | | | | |
|---|---|---|---|---|---|
| | tope | 10\hat{ }7(VSV-MARV-GP) | 1 (VSV-EBOV-GP) | 10(VSV-EBOV-GP) | 10\hat{ }6(VSV-EBOV-GP) |
| Epi- | Se- | NHP13-NHP16 | NHP9-NHP12 | NHP5-NHP8 | NHP1-NHP4 |

| tope | quence | Protein | D-28 | D-14 | D0 | D6 | D-28 | D6 | D28 | D-28 | D-14 | D0 | D6 | D28 | D-28 | D-14 | D0 | D6 | D28 |
|---|---|---|---|---|---|---|---|---|---|---|---|---|---|---|---|---|---|---|---|
| VSV1 | EDPVE | Nucleo- | 0/4 | 0/4 | 0/4 | 2/4 | 0/4 | 1/4 | 0/3 | 0/4 | 0/4 | 0/4 | 2/4 | 1/4 | 0/4 | 0/4 | 0/4 | 0/4 | 2/4 |
| | YPADY | capsid- | (0%) | (0%) | (0%) | (50%) | (0%) | (251%) | (0%) | (0%) | (0%) | (0%) | (50%) | (25%) | (0%) | (0%) | (0%) | (0%) | (50%) |
| | FRKSK | protein | | | | | | | | | | | | | | | | | |
| | EI | | | | | | | | | | | | | | | | | | |
| VSV2 | FDVWG | Nucleo- | 0/4 | 0/4 | 0/4 | 1/4 | 1/4 | 1/4 | 1/3 | 0/4 | 0/4 | 0/4 | 4/4 | 3/4 | 0/4 | 0/4 | 0/4 | 2/4 | 4/4 |
| | NDSNY | capsid- | (0%) | (0%) | (0%) | (25%) | (25%) | (25%) | (33%) | (0%) | (0%) | (0%) | (100%) | (75%) | (0%) | (0%) | (0%) | (50%) | (100%) |
| | TKIV | protein | | | | | | | | | | | | | | | | | |
| VSV3 | LFQED | Phospho- | 0/4 | 0/4 | 0/4 | 2/4 | 0/4 | 0/4 | 0/3 | 0/4 | 0/4 | 0/4 | 0/4 | 0/4 | 0/4 | 0/4 | 0/4 | 4/4 | 4/4 |
| | GVEEH | protein | (0%) | (0%) | (0%) | (50%) | (0%) | (0%) | (0%) | (0%) | (0%) | (0%) | (0%) | (0%) | (0%) | (0%) | (0%) | (100%) | (100%) |
| | TKPSY | | | | | | | | | | | | | | | | | | |
| | FQAAD | | | | | | | | | | | | | | | | | | |
| | DS | | | | | | | | | | | | | | | | | | |
| VSV4 | AEQVE | Phospho- | 0/4 | 0/4 | 0/4 | 3/4 | 0/4 | 1/4 | 2/3 | 0/4 | 0/4 | 0/4 | 2/4 | 2/4 | 0/4 | 0/4 | 0/4 | 3/4 | 4/4 |
| | GFIQG | protein | (0%) | (0%) | (0%) | (75%) | (0%) | (25%) | (66%) | (0%) | (0%) | (0%) | (75%) | (75%) | (0%) | (0%) | (0%) | (75%) | (100%) |
| | PLDDY | | | | | | | | | | | | | | | | | | |
| | ADEE | | | | | | | | | | | | | | | | | | |
| VSV5 | GKKSK | Matrix- | 0/4 | 0/4 | 0/4 | 3/4 | 0/4 | 1/4 | 2/3 | 0/4 | 0/4 | 0/4 | 2/4 | 4/4 | 0/4 | 0/4 | 0/4 | 4/4 | 4/4 |
| | KLGIA | protein | (0%) | (0%) | (0%) | (75%) | (0%) | (25%) | (66%) | (0%) | (0%) | (0%) | (75%) | (100%) | (0%) | (0%) | (0%) | (100%) | (100%) |
| | PPPYE | | | | | | | | | | | | | | | | | | |
| | EDTSM | | | | | | | | | | | | | | | | | | |
| | EYA | | | | | | | | | | | | | | | | | | |
| VSV61 | PTAAQ | Pol- | 0/4 | 0/4 | 0/4 | 0/4 | 0/4 | 1/4 | 1/3 | 0/4 | 0/4 | 0/4 | 1/4 | 2/4 | 0/4 | 0/4 | 0/4 | 3/4 | 4/4 |
| | VQDFG | ymer- | (0%) | (0%) | (0%) | (0%) | (0%) | (25%) | (33%) | (0%) | (0%) | (0%) | (25%) | (75%) | (0%) | (0%) | (0%) | (75%) | (100%) |
| | DKWHE | ase | | | | | | | | | | | | | | | | | |
| | LPL | | | | | | | | | | | | | | | | | | |
| | VSV7M | Pol- | 0/4 | 0/4 | 0/4 | 0/4 | 0/4 | 0/4 | 1/3 | 0/4 | 0/4 | 0/4 | 1/4 | 1/4 | 0/4 | 0/4 | 0/4 | 1/4 | 3/4 |
| | RVHNN | ymer- | (0%) | (0%) | (0%) | (0%) | (0%) | (0%) | (0%) | (0%) | (0%) | (0%) | (25%) | (25%) | (0%) | (0%) | (0%) | (25%) | (75%) |
| | TLINS | ase | | | | | | | | | | | | | | | | | |
| | TSQRV | | | | | | | | | | | | | | | | | | |
| VSV8 | NTAVK | Pol- | 0/4 | 0/4 | 0/4 | 0/4 | 0/4 | 0/4 | 2/3 | 0/4 | 0/4 | 0/4 | 0/4 | 4/4 | 1/4 | 0/4 | 0/4 | 3/4 | 3/4 |
| | VLAQG | ymer- | (0%) | (0%) | (0%) | (0%) | (0%) | (0%) | (66%) | (0%) | (0%) | (0%) | (0%) | (100%) | (0%) | (0%) | (0%) | (75%) | (75%) |
| | DNQVI | ase | | | | | | | | | | | | | | | | | |
| | CTQYK | | | | | | | | | | | | | | | | | | |
| | T | | | | | | | | | | | | | | | | | | |
| VSV9 | KIGTG | Pol- | 0/4 | 0/4 | 0/4 | 0/4 | 0/4 | 4/4 | 3/3 | 0/4 | 0/4 | 0/4 | 4/4 | 4/4 | 0/4 | 0/4 | 0/4 | 4/4 | 4/4 |
| | KLGLL | ymer- | (0%) | (0%) | (0%) | (0%) | (0%) | (100%) | (100%) | (0%) | (0%) | (0%) | (100%) | (100%) | (0%) | (0%) | (0%) | (100%) | (100%) |
| | INDDE | ase | | | | | | | | | | | | | | | | | |
| | TM | | | | | | | | | | | | | | | | | | |

### VSV-EBOV-GP Vaccinated Human Subjects

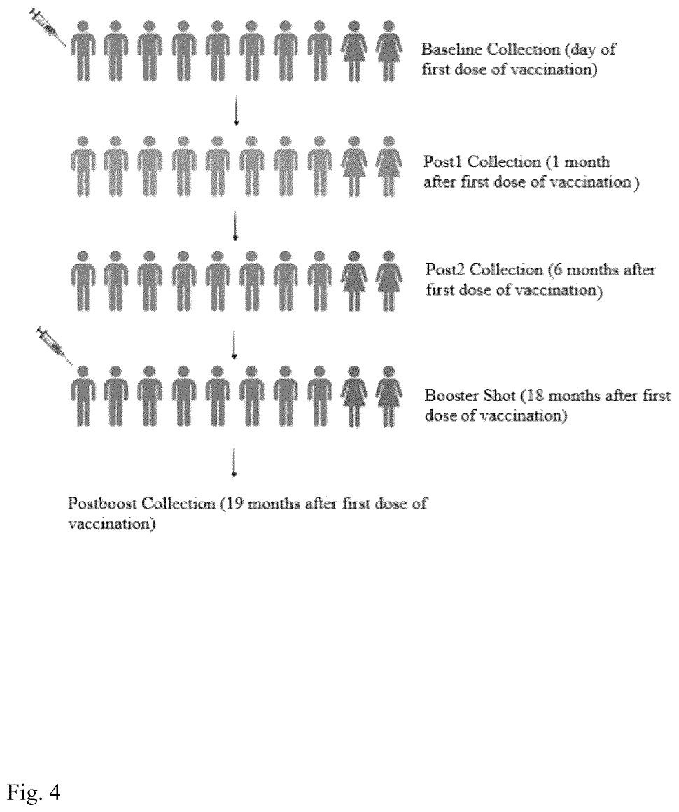

Plasma samples were collected from 10 human subjects (8 males and 2 females) vaccinated with VSV-EBOV-GP vaccine prior to vaccination (Baseline), 1 month after receiving the first dose of vaccine (Post 1), 6 months after receiving the first dose first dose vaccine (Post 2), and either one month after receiving the second dose of vaccine or 19 months after receiving the first dose of vaccine (Post boost) (FIG. 4). Immunoreactive epitopes found in humans were common to the those identified in monkeys (Table 5).

TABLE 5
Immunoreactive epitopes in EBOV-GP and sGP detected using plasma from
humans immunized with VSV-EBOV-Makona-GP. (SEQ ID NOS: 46-57, from top to bottom).

| Epitope | Epitope Sequence | Protein | Baseline | Post-1 | Post-2 | Postboost |
|---|---|---|---|---|---|---|
| MGP1 | LILPQAKKDFFSSHPLREPVNATEDP | GP1, 2 | 0/10(0%) | 1/10(10%) | 0/10(0%) | 4/10(40%) |
| MGP2 | TTGKLIWKVNPEIDTTIGEW | GP1, 2 | 0/10(0%) | 0/10(0%) | 0/10(0%) | 6/10(60%) |
| MGP3 | EIDTTIGEWAFWETKKNLTRKIR | GP1, 2 | 0/10(0%) | 3/10(30%) | 1/10(10%) | 10/10(100%) |
| MGP4 | AVSNGPKNISGQSPARTSSDPETNTTNE | GP1, 2 | 0/10(0%) | 7/10(70%) | 4/10(40%) | 7/10(70%) |
| MGP5 | NTTNEDHKIMASENSSAMV | GP1, 2 | 0/10(0%) | 1/10(10%) | 1/10(10%) | 8/10(80%) |
| MGP6 | ASENSSAMVQVHSQGRKAAVSHLTTLAT | GP1, 2 | 0/10(0%) | 1/10(10%) | 0/10(0%) | 7/10(70%) |
| MGP7 | DNSTHNTPVYKLDISEATQVGQHHRRAD | GP1, 2 | 0/10(0%) | 8/10(80%) | 6/10(60%) | 10/10(100%) |
| | NDSTASDTPPA | | | | | |
| MGP8 | PATTAAGPLKAENTNTSKSADSLDL | GP1, 2 | 2/10(20%) | 3/10(30%) | 3/10(30%) | 5/10(50%) |
| MGP9 | ETAGNNNTHHQDTGEESASSGKLGLITN | GP1, 2 | 0/10(0%) | 7/10(70%) | 4/10(40%) | 10/10(100%) |
| | TIAGVAGLITGGRRTRREVI | | | | | |
| MGP12 | DKIDQIIHDFVDKTLPDQGDNDNWWTGW | GP1, 2 | 1/10(10%) | 4/10(40%) | 3/10(30%) | 8/10(80%) |
| | RQW | | | | | |
| MSGP1 | FLILPQAKKDFFSSHPLREPVNATEDP | SGP | 0/10(10%) | 1/10(10%) | 0/10(10%) | 4/10(40%) |
| MSGP3 | EIDTTIGEWAFWETKKSTLE | sGP | 0/10(10%) | 1/10(10%) | 1/10(10%) | 9/10(90%) |

4 VSV epitopes were identified in human VSV-EBOV-GP vaccinated samples, with 2 epitopes in matrix protein. Epitope VSV1 has maximum immunoreactivity in post-2 samples, VSV10 epitope has immunoreactivity in 50% of samples or above at post-1 and postboost. Matrix protein epitopes had maximum immunoreactivity in post-boost. Epitope VSV5 was also present in NHP samples and can be used as a serological marker for vaccination (Table 6).

TABLE 6
The table lists the immunoreactive epitopes of VSV vector from VSV-
EBOV-GP vaccinated humans. (SEQ ID NOS: 58-61, from top to bottom).

| Epitope | Epitope Sequence | Protein | Baseline | Post1 | Post2 | Postboost |
|---|---|---|---|---|---|---|
| VSV1 | PANEDPVEYP | Nucleoocapsid | 0/10 (0%) | 1/10 (10%) | 6/10 (60%) | 3/10 (30%) |
| | ADYFRKSKEI | protein | | | | |
| VSV5 | GKKSKKLGIA | Matrix protein | 0/10 (0%) | 4/10 (40%) | 4/10 (40%) | 8/10 (80%) |
| | PPPYEEDTSM | | | | | |
| | EYA | | | | | |
| VSV10 | AVGEIDEIEA | Phosphoprotein | 0/10 (0%) | 6/10 (60%) | 0/10 (0%) | 5/10 (50%) |
| | QRAEKSNYEL | | | | | |
| | FQEDGVE | | | | | |
| VSV11 | SMEYAPSAPI | Matrix protein | 0/10 (0%) | 2/10 (20%) | 1/10 (10%) | 5/10 (50%) |
| | DKSYFGVDEM | | | | | |
| | DTYDPNQ | | | | | |

### Example 3—Utility of EBOV GP-sGP Epitope and VSV Epitope Peptides

To examine utility of a few selected identified peptides for diagnostic assays, MGP3 DTTIGEWAFWETKKNLTRK {LYS (BIOTIN}) (SEQ ID NO: 63) and VSV5 (GKKSKKLGIAPPPYEEDTS {LYS (BIOTIN}) (SEQ ID NO: 64) peptides were synthesized with biotinylation on C-Terminal for a simple peptide ELISA system. Peptides were modified at N-terminal with acetylation and at C-terminal with biotinylation. NHP and human serum samples from vaccines were used at 1:100 dilution and established streptavidin-biotinylated ELISAs for both the peptides. Anti-macaque secondary IgG antibodies with HRP conjugate and anti-human secondary IgG antibodies with HRP conjugate were used to detect reactivity for NHP and human samples respectively.

### ELISA Results for NHP Samples:

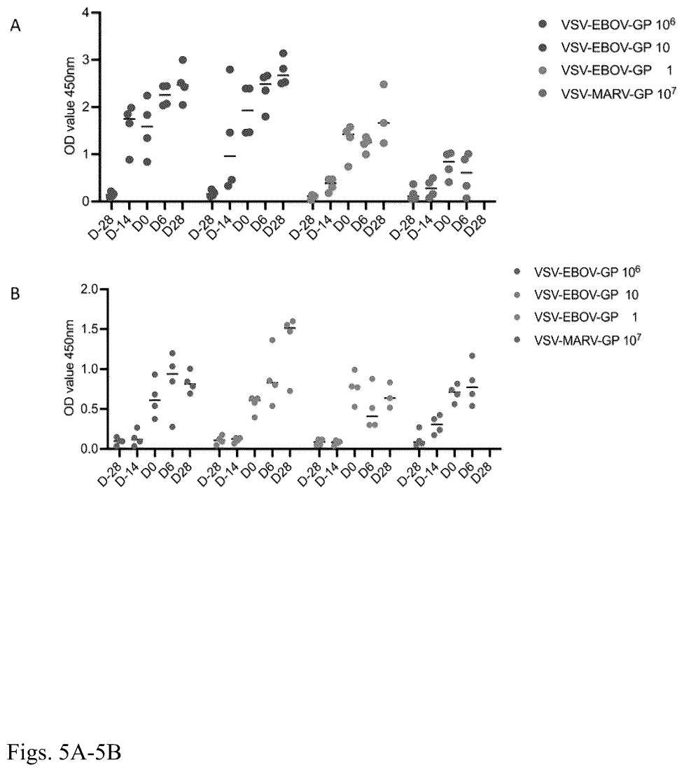

ELISA results showed gradual increase in reactivity (OD readings at 450 nm) to both peptides in samples with low titer of vaccination (VSV-EBOV-GP dose 1), to (VSV-EBOV-GP dose 10) to maximum reactivity at highest titer of vaccination (VSV-EBOV-GP 10⁶). Control samples from NHP inoculated with Marburg virus vaccine (VSV-MARV-GP 10⁷) showed minimal reactivity to MGP3 peptide, but similar reactivity with VSV5 peptide (FIG. 5A, FIG. 5B). Highest IgG reactivity was recorded at D6 and D28 at respective doses of vaccination. MGP3 peptide showed higher reactivity after infection (challenge) with EBOV-Makona in all NHP.

### ELISA Results: Human Vaccine Samples:

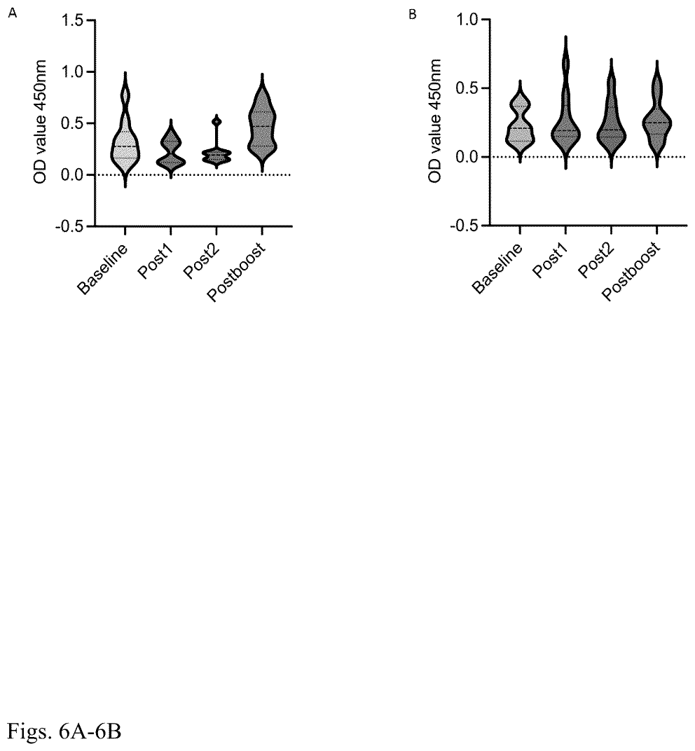

Human samples had overall low reactivity compared to NHPs (FIG. 6). MGP3 peptide showed higher reactivity after post-boost in all samples from vaccinated individuals. MGP3 peptide reactivity increased from baseline to post 1 month, decreased at post 6 month and increased at highest at post boost time point.

### Summary of Findings from Examples 2 and 3

Sixteen EBOV peptides (MGP1-MSGP4) listed in Table 2 and 19 EBOV peptides (MNP1-ML1) listed in Table 3 can be used to identify people who have been infected with EBOV.

Sixteen EBOV peptides (MGP1-MSGP4) listed in Table 2, in combination with 9 VSV peptides (VSV1-VSV9) listed in Table 4 and 2 additional VSV peptides (VSV10 and VSV11) listed in Table 6 can be used to identify people who have been Vaccinated.

Sixteen EBOV peptides (MGP1-MSGP4) listed in Table 2 and 19 EBOV peptides (MNP1-ML1) listed in Table 3 can also identify the EBOV survivors.

Combination of 16 EBOV peptides (MGP1-MSGP4) listed in Table 2, 19 EBOV peptides (MNP1-ML1) listed in Table 3, 9 VSV peptides (VSV1-VSV9) listed in Table 4, and 2 additional VSV peptides (VSV10 and VSV11) can be used to determine individuals who received vaccination and had a past natural infection or both.

### Example 4—Utility of Conserved Domain Peptide Identified in GP and sGP

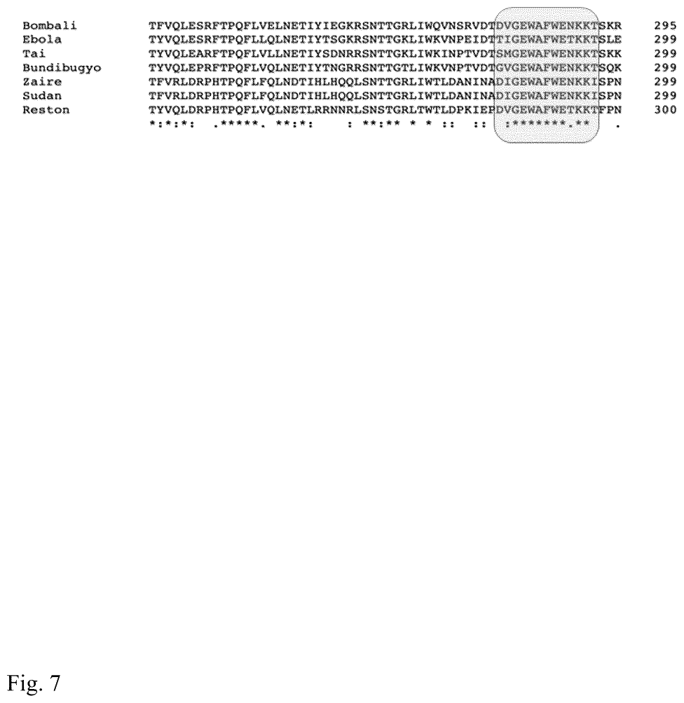

In this study, we have discovered immunoreactive epitopes of most interest to be MGP3-EIDTTIGEWAFWETKKNLTRKIR (SEQ ID NO: 4), MGP1-LILPQAKKDFFSSHPLREPVNATEDP (SEQ ID NO: 2) and MGP12-DKIDQIIHDFVDKTLPDQGDNDNWWTGWRQ (SEQ ID NO: 13). MGP3 has an immunoreactive core region as TIGEWAFWETKKNLT (SEQ ID NO: 1) and MGP1 has an immunoreactive core region LPQAKKDFFSSHPLRE (SEQ ID NO: 62). The epitope core region of MGP3 TIGEWAFWETKKNLT (SEQ ID NO: 1) is most important if used as diagnostic marker because of its presence in both sGP and ssGP makes it abundant and easily accessible to circulating antibodies in patient samples, owing to high abundance of sGP production and its extracellular release (FIG. 7). In addition to such attributes this epitope is also surface exposed as depicted in FIG. 6 representing EBOV-GP as monomer and EP1 as green, further making it a strong candidate for diagnosis. Interestingly, this epitope is also conserved among other ebolaviruses as shown in ClustalW alignment, making it a conserved epitope which can be used to detect all the EBOV species, other known filoviruses, and probably filoviruses yet to be discovered. Also, the epitope MGP12 lies near to the transmembrane region at the C-terminal. MGP12 belongs to the region MPER which has been reported as a region which provides protection in survivors against EBOV (FIG. 7).

### Validation of Immunoreactive Peptides in a Low Density PEPperPRINT Arrays:

We identified most reactive peptide regions from data presented above, and 120 peptides (16 amino acids) in duplicate from all peptide regions; were printed on a low-density peptide microarray to distinguish and differentially diagnose individuals with prior EBOV immunization from EBOV recovered patients who had Ebola virus disease in the past. The concept lies in measuring immunoreactivity to peptides of non-structural proteins in EBOV recovered patients only, immunoreactivity to structural proteins in immunized and recovered patients both, immunoreactivity to VSV peptides in immunized individuals only, and immunoreactivity to peptides of structural proteins, peptides of nonstructural proteins and VSV peptides in individuals who had been immunized by EBOV vaccine and had prior exposure to Ebola virus both. In this low-density array 9 replicates influenza hemagglutinin (YPYDVPDYAG) (SEQ ID NO: 65) and 8 replicates of polio virus (KEVPALTAVETGAT), (SEQ ID NO: 66) were also added as positive control peptides.

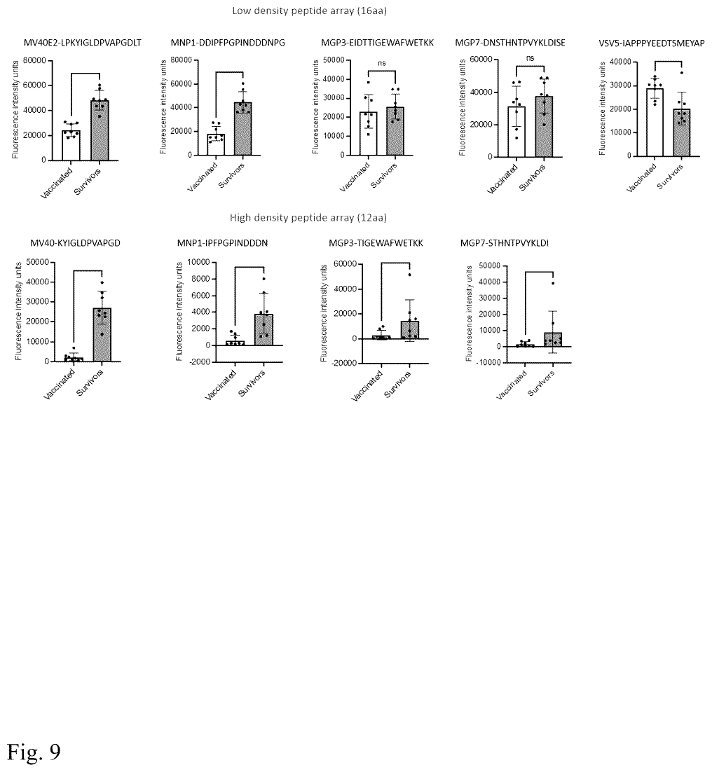

We measured antibodies to these peptides in serum samples from 16 individuals, among those 8 were from rVSV-ZEBOV-vaccinated and 8 were from EBOV infected survivors. We found significantly differential immune responses among immunized and survivor group for MV40E2, MNP1, MGP3, MGP7, VSV5 peptide regions. MGP3 and MGP7 peptides from glycoprotein (structural protein) and MV40E2 peptide from VP40 (non-structural protein) were highly reactive in EBOV infected survivors. MGP3 and MGP7 peptides from glycoprotein (structural protein) and VSV5 peptide from vaccine construct of VSIV were highly reactive in rVSV-ZEBOV-vaccinated individuals (FIG. 9, upper panel). All these peptide regions except VSV5 were similarly immunoreactive in high-density arrays as described earlier and matched data in low-density array (FIG. 9, lower panel). However, VSV5 showed better reactivity in low-density arrays.

We can use panel of these 5 peptides (MV40E2, MNP1, MGP3, MGP7, VSV5) to differentially diagnose immunized versus EBOV survivors.

### REFERENCES

1. Buus, S., et al., High-resolution mapping of linear antibody epitopes using ultrahigh-density peptide microarrays. Mol Cell Proteomics, 2012. 11 (12): p. 1790-800.
2. Mishra N, Huang X, Joshi S, Guo C, Ng J, Thakkar R, Wu Y, Dong X, Li Q, Pinapati R S, Sullivan E, Caciula A, Tokarz R, Briese T, Lu J, Lipkin W I. Immunoreactive peptide maps of SARS-CoV-2. Commun Biol. 2021 Feb. 12; 4(1):225. doi: 10.1038/s42003-021-01743-9. Erratum in: Commun Biol. 2021 Mar. 9; 4(1):339. PMID: 33580175; PMCID: PMC7881038

1. A platform comprising at least one peptide that is reactive with one or more primate antibodies associated with natural infection by EBOV, wherein the at least one peptide comprises an amino acid sequence in the group consisting of SEQ ID NOS: 2-17 and SEQ ID NOS: 18-36 or a portion thereof;
or
a platform comprising at least two peptides that are each reactive with one or more primate antibodies associated with vaccination against EBOV by a vesicular stomatitis virus-based vaccine, wherein a first peptide of the at least two peptides comprises an amino acid sequence in the group consisting of SEQ ID NOS: 2-17 or a portion thereof, and wherein a second peptide of the at least two peptides comprises an amino acid sequence in the group consisting of SEQ ID NOS: 37-45 and SEQ ID NOS: 58-61 or a portion thereof;
or
a platform comprising (a) at least two peptides that are each reactive with one or more primate antibodies associated with vaccination against EBOV by a vesicular stomatitis virus-based vaccine, and (b) at least one peptide that is reactive with one or more primate antibodies associated with natural infection by EBOV, wherein the at least two peptides of (a) comprise (i) a first peptide comprising an amino acid sequence in the group consisting of SEQ ID NOS: 2-17 or a portion thereof, and (ii) a second peptide comprises an amino acid sequence in the group consisting of SEQ ID NOS: 37-45 and SEQ ID NOS: 58-61 or a portion thereof, and wherein the at least one peptide of (b) comprises an amino acid sequence chosen from the group consisting of the sequences listed in SEQ ID NOS: 2-17 and the sequences listed in SEQ ID NOS: 18-36 or a portion thereof.

2. A method of identifying a subject who has been infected with EBOV, comprising:
a. contacting a sample from the subject with a platform described herein, under conditions sufficient to allow binding of antibody(ies) in the sample to peptide(s) of the platform so as to form an antibody-peptide complex; and
b. detecting any antibody-peptide complex so formed;
wherein detection of the complex is indicative of an antibody associated with a EBOV natural infection being present in the sample and identifies the subject as being previously infected with EBOV;
or
a method of identifying a subject who has been vaccinated against EBOV, comprising:
a. contacting a sample from the subject with a platform described herein, under conditions sufficient to allow binding of antibody(ies) in the sample to the first peptide, and to allow binding of antibody(ies) in the sample to the second peptide of the platform so as to form a first antibody-peptide complex and a second antibody-peptide complex, respectively; and
b. detecting any antibody-peptide complexes so formed;
wherein detection of both a first antibody-peptide complex and of a second antibody-peptide complex is indicative of antibodies associated with vaccination against EBOV with a vesicular stomatitis virus-based vaccine being present in the sample and identifies the subject as being previously vaccinated with an EBOV vesicular stomatitis virus-based vaccine;
or
a method of identifying a subject who has been infected with EBOV and/or vaccinated against EBOV, comprising:
a. contacting a sample from the subject with a platform described herein, under conditions sufficient to (a) allow binding of antibody(ies) in the sample to the first peptide comprising an amino acid sequence of at least five consecutive amino acids of a sequence in the group consisting of SEQ ID NOS: 2-17, and allow binding of antibody(ies) in the sample to the second peptide comprising an amino acid sequence of at least five consecutive amino acids of a sequence in the group consisting of SEQ ID NOS: 37-45 and SEQ ID NOS: 58-61, and also (b) allow binding of antibody(ies) in the sample to a third peptide comprising an amino acid sequence chosen from the group consisting of the sequences listed in SEQ ID NOS: 2-17 and SEQ ID NOS: 18-36, so as to permit formation of (a) a first antibody-first peptide complex and a second antibody-second peptide complex and/or (b) a third antibody-third peptide complex, respectively; and
b. detecting any antibody-peptide complexes so formed;
wherein detection of both a first antibody-first peptide complex and of a second antibody-second peptide complex is indicative of antibodies associated with vaccination against EBOV with a vesicular stomatitis virus-based vaccine being present in the sample and identifies the subject as being previously vaccinated with an EBOV vesicular stomatitis virus-based vaccine, and wherein detection of a third antibody-third peptide complex is indicative of antibodies associated with natural EBOV infection being present in the sample.

3. A kit comprising a platform of claim 1 and one or more detectably-labeled antibodies or fragments thereof which bind a primate antibody.

4. The kit of claim 3, wherein the detectably-labeled antibodies or fragments thereof are labeled with an enzyme.

5. The kit of claim 3, wherein the one or more detectably-labeled antibodies or fragments thereof bind a human IgG antibody or a human IgM antibody.

6. The kit of claim 5, wherein the one or more detectably-labeled antibodies or fragments thereof bind a Fab of an human IgG antibody.

7. The kit of claim 3, further comprising a buffer solution, a wash solution, and/or an enzyme substrate or chromogen.

8. A peptide microarray comprising peptides that is reactive with, and specific for antibodies of natural infection by EBOV, wherein the peptides comprise the amino acid sequence chosen from the group consisting of the sequences listed in Table 2 and the sequences listed in Table 3 and combinations thereof and/or a collection or set of peptides shifted at least one residue across at least one peptide, wherein the peptide comprises the amino acid sequence chosen from the group consisting of the sequences listed in Table 2 and the sequences listed in Table 3 and combinations thereof;
or
a peptide microarray comprising peptides that are reactive with, and specific for antibodies of vaccination against EBOV, wherein the peptides comprise the amino acid sequence chosen from the group consisting of the sequences listed in Table 2 and the sequences listed in Table 4 and the sequences listed in Table 6 and combinations thereof and/or a collection or set of peptides shifted at least one residue across at least one peptide, wherein the peptide comprises the amino acid sequence chosen from the group consisting of the sequences listed in Table 2 and the sequences listed in Table 4 and the sequences listed in Table 6 and combinations thereof.

9. The platform of claim 1, comprising a solid phase or semi-solid phase to which the peptides are attached, adsorbed, immobilized, bound or enclosed fully or partially.

10. The platform of claim 1, which is an ELISA plate or ELISA plate coating, a microarray, a lateral flow device, a Western blot membrane or gel, a radioimmunoassay, or a bead of a bead-based assay.

11. The platform of claim 1, wherein the solid phase comprises a plastic polymer or a glass.

12. The method of claim 2, wherein the subject is a human subject.

13. The method of claim 2, wherein the subject is a test subject who has been administered a vaccine or immunomodulatory agent.

14. The method of claim 2, wherein the sample is chosen from the group consisting of nasopharyngeal aspirate, blood, cerebrospinal fluid, saliva, serum, plasma, urine, sputum, bronchial lavage, pericardial fluid, and peritoneal fluid.

15. The method of claim 2, wherein the sample is chosen from the group consisting of cells, cell culture, cell culture medium and compositions used for the development of pharmaceutical and therapeutic agents.
